(19) United States
(12) Patent Application Publication
(10) Pub. No.: US 2023/0183813 A1
(43) Pub. Date: Jun. 15, 2023

(54) DETECTING CYTOGENETICS USING LIQUID BIOPSY

(71) Applicant: GLM INNOVATIONS, LLC, Edison, NJ (US)

(72) Inventor: Maher Albitar, Valley Center, CA (US)

(73) Assignee: GLM INNOVATIONS, LLC, Edison, NJ (US)

(21) Appl. No.: 18/077,910

(22) Filed: Dec. 8, 2022

Related U.S. Application Data
(60) Provisional application No. 63/288,147, filed on Dec. 10, 2021.

(51) Int. Cl. C12Q 1/6886; G16B 35/20

(52) U.S. Cl. CPC C12Q 1/6886; G16B 35/20; C12Q 2600/112; C12Q 2600/106

(57) ABSTRACT

A method of determining copy number variation of chromosomes and genes in a sample from a subject having cancer or suspected of having cancer and of determining diagnosis, prognosis, and potential therapy when compared to a reference sample.

### CROSS-REFERENCE TO RELATED APPLICATIONS

This application claims the benefit of priority to U.S. Provisional Application No. 63/288,147 (filed Dec. 10, 2021), which is incorporated herein by reference in its entirety.

### FIELD OF THE DISCLOSURE

The present disclosure relates to methods of determining a prognosis for a subject having cancer or suspected of having cancer by determining copy number variation of genomic segments.

### BACKGROUND OF THE INVENTION

Chromosomal variations play a major role in the diagnosis, prognosis and selection of therapy in hematologic neoplasms. Cytogenetic studies are recommended for almost all myeloid neoplasms and most lymphoid neoplasms. Cytogenetic studies require fresh viable cells for culturing, and performing cytogenetics analysis remains mainly manual, expensive and time consuming. Fluorescence in situ hybridization (FISH) studies help in reducing time and can be used in formalin-fixed paraffin-embedded (FFPE) samples. However, FISH can test for predetermined chromosomal abnormalities one abnormality at a time. Recent advances in high-throughput genomic technologies allowed broader evaluation of chromosomal abnormalities using arrays. Array technology offers wider genome coverage with higher resolution. However, this technology requires significant quantity of samples. Next-generation sequencing (NGS) is increasingly being implemented to evaluate chromosomal structural abnormalities including chromosomal translocations. Whole-genome sequencing has been shown to be reliable in detecting various chromosomal abnormalities including amplifications, copy number variations (CNVs), uniparental disomy (meaning the condition of having a chromosome represented twice in a chromosomal complement), mosaicism (meaning a condition in which cells within the same individual have a different genetic makeup), small (indels) and single nucleotide variations (SNVs).

However, the high cost of whole-genome sequencing restricts the use of this approach and currently the use of targeted sequencing panels for detecting chromosomal structural abnormalities is feasible. Furthermore, targeted sequencing has the advantage of being a practical and cost-effective approach for liquid biopsy for analyzing cell-free DNA (cfDNA).

The clinical reliability of liquid biopsies in detected SNVs has been established and accepted in certain circumstances. The clinical relevance of detecting chromosomal structural abnormalities in cancer has not been addressed adequately. Detecting chromosomal structural abnormalities has been studied in prenatal diagnosis or so called NIPT (Noninvasive Prenatal Testing) and it is a currently acceptable approach for pre-natal screening. However, NIPT is used mainly for detecting trisomies in specific chromosomes and not for potential small size abnormalities.

We explored the potential of evaluating chromosomal structural abnormalities in liquid biopsy.

### SUMMARY OF THE INVENTION

According to one aspect, the present disclosure provides a method for identifying a subject diagnosed with cancer or suspected of having cancer as falling within a treatable prognostic group, the method comprising:
a) providing a biological sample from the subject;
b) determining copy number of one or more target genes or fragments of the target genes compared to a reference sample by:
1) preparing a cell-free DNA (cfDNA) sample from the biological sample of step a);
2) preparing a sequencing library from the cfDNA from the biological sample, wherein preparing the library comprises consecutive steps of fragmenting the cfDNA, end-repairing, dA-tailing and adaptor ligating the cfDNA fragments;
3) sequencing the adaptor-ligated cfDNA fragments of the one or more target genes or fragments thereof from the biological sample;
4) determining copy number of the one or more target genes or fragments thereof from the sequences of the adaptor-ligated cfDNA fragments from the biological sample;
5) comparing the copy number of the one or more target genes or cfDNA fragments thereof for the biological sample with that of a reference sample, wherein the comparison can determine one or more chromosomal abnormalities in the biological sample;
   c) identifying the prognostic group of the subject based on step b), wherein the presence of one or more chromosomal abnormalities in the one or more target genes or fragments thereof of the biological sample when compared to the reference sample indicates a subject having cancer with an adverse, intermediate, or favorable prognosis; and
   d) qualifying the subject for chemotherapy or immunotherapy where the results in (c) indicate an adverse, intermediate or favorable prognosis.

According to some embodiments, the biological sample is a tissue biopsy of the cancer or a liquid biopsy. According to some embodiments, the biological sample is blood, plasma, serum, urine, stool, saliva, tissue, or bodily fluid. According to some embodiments, the biological sample is plasma derived from peripheral blood that comprises a mixture of cfDNA derived from normal and cancerous cells.

According to some embodiments, the one or more target genes or fragments thereof are selected from Table 3.

According to some embodiments, the target gene or fragments thereof is protein-coding regions of a gene.

According to some embodiments, sequencing the adaptor-ligated cfDNA fragments of the one or more target genes or fragments thereof from the biological sample includes employing a next generation sequencing (NGS) method.

According to some embodiments, the cancer is renal carcinoma. According to some embodiments, the cancer is colorectal carcinoma. According to some embodiments, the cancer is skin cancer. According to some embodiments, the cancer is myelodysplastic syndrome (MDS). According to some embodiments, the cancer is leukemia. According to some embodiments, the cancer is lymphoma. According to some embodiments, the cancer is myeloma. According to some embodiments, the cancer is tumors of the central nervous system. According to some embodiments, the cancer is breast cancer. According to some embodiments, the cancer is prostate cancer. According to some embodiments, the cancer is cervical cancer. According to some embodiments, the cancer is uterine cancer. According to some embodiments, the cancer is lung cancer. According to some embodiments, the cancer is ovarian cancer. According to some embodiments, the cancer is testicular cancer. According to some embodiments, the cancer is thyroid cancer. According to some embodiments, the cancer is astrocytoma. According to some embodiments, the cancer is glioma. According to some embodiments, the cancer is pancreatic cancer. According to some embodiments, the cancer is mesotheliomas. According to some embodiments, the cancer is gastric cancer. According to some embodiments, the cancer is liver cancer. According to some embodiments, the cancer is renal cancer. According to some embodiments, the cancer is nephroblastoma. According to some embodiments, the cancer is bladder cancer. According to some embodiments, the cancer is oesophageal cancer. According to some embodiments, the cancer is cancer of the larynx. According to some embodiments, the cancer is cancer of the parotid. According to some embodiments, the cancer is cancer of the biliary tract. According to some embodiments, the cancer is endometrial cancer. According to some embodiments, the cancer is adenocarcinomas. According to some embodiments, the cancer is small cell carcinomas. According to some embodiments, the cancer is neuroblastomas. According to some embodiments, the cancer is adrenocortical carcinomas. According to some embodiments, the cancer is epithelial carcinomas. According to some embodiments, the cancer is desmoid tumors. According to some embodiments, the cancer is desmoplastic small round cell tumors. According to some embodiments, the cancer is endocrine tumors. According to some embodiments, the cancer is Ewing sarcoma family tumors. According to some embodiments, the cancer is germ cell tumors. According to some embodiments, the cancer is hepatoblastomas. According to some embodiments, the cancer is hepatocellular carcinomas. According to some embodiments, the cancer is non-rhabdomyosarcome soft tissue sarcomas. According to some embodiments, the cancer is osteosarcomas. According to some embodiments, the cancer is peripheral primitive neuroectodermal tumors. According to some embodiments, the cancer is retinoblastomas. According to some embodiments, the cancer is rhabdomyosarcomas.

According to some embodiments, the chromosomal abnormalities in the one or more target genes or fragments thereof is a deletion, a duplication, or a combination thereof

According to some embodiments, the subject is a human.

According to some embodiments, step b-4) comprises determining sequence read depth of one or more target segments and one or more non-target segments.

According to some embodiments, the reference sample is a sample from one or more subjects with a cancer or a sample from one or more subjects not with a cancer, or both.

According to some embodiments, qualifying the subject for chemotherapy or immunotherapy comprise identifying the subject as a candidate for chemotherapy or immunotherapy based on the adverse, intermediate or favorable prognosis.

According to some embodiments, qualifying the subject for chemotherapy or immunotherapy comprises one or more of: 1

displaying on a graphical user interface an identification of the subject as a candidate for chemotherapy or immunotherapy;
storing data identifying the subject as a candidate for chemotherapy or immunotherapy;
sending an electronic communication including an identification of the subject as a candidate for chemotherapy or immunotherapy;
displaying on a graphical user interface a recommendation of chemotherapy or immunotherapy for the subject;
storing data including a recommendation of immunotherapy or chemotherapy for the subject; and
sending an electronic communication including a recommendation of immunotherapy or chemotherapy for the subject.

According to some embodiments, identifying the prognostic group of the subject as adverse includes the biological sample comprising one or more chromosomal abnormalities selected from the group consisting of t(6;9)(p23;q34); DEK-NUP214; t(v;11q23); KMT2A rearranged; t(9;22)(q34;q11); BCR-ABL1; inv(3)(q21q26) or t(3;3)(q21;q26); GATA2,MECOM(EVI1); del(5q); abn(17p); complex karyotype; and monosomal karyotype.

According to some embodiments, identifying the prognostic group of the subject as adverse includes the biological sample comprising one or more chromosomal abnormalities selected from the group consisting of t(6;9)(p23;q34.1); DEK-NUP214; t(v;11q23.3); KMT2A rearranged; t(9;22)(q34.1;q11.2); BCR-ABL1; inv(3)(q21.3q26.2) or t(3;3)(q21.3;q26.2); GATA2,MECOM(EVI1); del(5q); abn(17p); complex karyotype; and monosomal karyotype.

According to some embodiments, identifying the prognostic group of the subject as favorable includes the biological sample comprising one or more chromosomal abnormalities selected from the group consisting of t(8;21)(q22;q22); RUNX1-RUNX1T1; inv(16)(p13q22) or t(16;16)(p13;q22); CBFB-MYH11; and t(15;17) PML-RARA; and does not include one or more chromosomal abnormalities selected from the group consisting of t(9;11)(p21;q23); MLLT3-KMT2A; t(6;9)(p23;q34); DEK-NUP214; t(v;11q23); KMT2A rearranged; t(9;22)(q34;q11); BCR-ABL1; inv(3)(q21q26) or t(3;3)(q21;q26); GATA2,MECOM(EVI1); del(5q); abn(17p); Complex karyotype; and monosomal karyotype.

According to some embodiments, identifying the prognostic group of the subject as favorable includes the biological sample comprising one or more chromosomal abnormalities selected from the group consisting of t(8;21)(q22;q22.1); RUNX1-RUNX1T1; inv(16)(p13.1q22) or t(16;16)(p13.1;q22); CBFB-MYH11; and t(15;17) PML-RARA; and does not include one or more chromosomal abnormalities selected from the group consisting of t(9;11)(p21.3;q23.3); MLLT3-KMT2A; t(6;9)(p23;q34.1); DEK-NUP214; t(v;11q23.3); KMT2A rearranged; t(9;22)(q34.1;q11.2); BCR-ABL1; inv(3)(q21.3q26.2) or t(3;3)(q21.3;q26.2); GATA2,MECOM(EVI1); del(5q); abn(17p); Complex karyotype; and monosomal karyotype.

According to some embodiments, identifying the prognostic group of the subject as intermediate includes the biological sample comprising one or more chromosomal abnormalities selected from the group consisting of t(9;11)(p21;q23); MLLT3-KMT2A; and does not include t(8;21)(q22;q22); RUNX1-RUNX1T1; inv(16)(p13q22) or t(16;16)(p13;q22); CBFB-MYH11; t(15;17) PML-RARA; t(6;9)(p23;q34); DEK-NUP214; t(v;11q23); KMT2A rearranged; t(9;22)(q34;q11); BCR-ABL1; inv(3)(q21q26) or t(3;3)(q21;q26); GATA2,MECOM(EVI1); del(5q); abn(17p); complex karyotype; and monosomal karyotype.

According to some embodiments, identifying the prognostic group of the subject as intermediate includes the biological sample comprising one or more chromosomal abnormalities selected from the group consisting of t(9;11)(p21.3;q23.3); MLLT3-KMT2A; and does not include t(8;21)(q22;q22.1); RUNX1-RUNX1T1; inv(16)(p13.1q22) or t(16;16)(p13.1;q22); CBFB-MYH11; t(15;17) PML-RARA; t(6;9)(p23;q34.1); DEK-NUP214; t(v;11q23.3); KMT2A rearranged; t(9;22)(q34.1;q11.2); BCR-ABL1; inv(3)(q21.3q26.2) or t(3;3)(q21.3;q26.2); GATA2,MECOM(EVI1); del(5q); abn(17p); complex karyotype; and monosomal karyotype.

According to one aspect, the present disclosure provides a method for treating a subject with a cancer, the method comprising:
a) providing a biological sample from the subject;
b) determining copy number of one or more target genes or fragments of the target genes compared to a reference sample by:
1) preparing a cell-free DNA (cfDNA) sample from the biological sample of step a);
2) preparing a sequencing library from the cfDNA from the biological sample, wherein preparing the library comprises consecutive steps of fragmenting the cfDNA, end-repairing, dA-tailing and adaptor ligating the cfDNA fragments;
3) sequencing the adaptor-ligated cfDNA fragments of the one or more target genes or fragments thereof from the sequences of the adaptor-ligated cfDNA fragments for the biological sample and determining copy number of the one or more target genes or fragments thereof;
4) comparing, by a computing system, the copy number of the one or more target genes or cfDNA fragments thereof for the biological sample with that of a reference sample, wherein the comparison can determine one or more chromosomal abnormalities in the biological sample;
   c) identifying the prognostic group of the subject based on step b), wherein the presence of one or more chromosomal abnormalities in the one or more target genes or fragments thereof for the biological sample when compared to the reference sample indicates a subject has a cancer with an adverse, intermediate, or favorable prognosis;
   d) qualifying the subject for adjunct therapy where the results in (c) indicate an adverse, intermediate or favorable prognosis, and
   e) administering a therapeutic amount of a chemotherapeutic or immunotherapeutic agent to the subject qualified for adjunct therapy in the adverse, intermediate or favorable prognosis group.

According to some embodiments, the biological sample is a tissue biopsy of the cancer or a liquid biopsy.

According to some embodiments, the biological sample is blood, plasma, serum, urine, stool, saliva, tissue, or bodily fluid.

According to some embodiments, the biological sample is plasma derived from peripheral blood that comprises a mixture of cfDNA derived from normal and cancerous cells.

According to some embodiments, the one or more target genes or fragments thereof are selected from Table 3.

According to some embodiments, the target gene or fragments thereof are a protein -coding regions of a gene.

According to some embodiments, sequencing the adaptor-ligated cfDNA fragments of the one or more target genes or fragments thereof from the biological sample includes employing a next generation sequencing (NGS) method.

According to some embodiments, the cancer is renal carcinoma. According to some embodiments, the cancer is colorectal carcinoma. According to some embodiments, the cancer is skin cancer. According to some embodiments, the cancer is myelodysplastic syndrome (MDS). According to some embodiments, the cancer is leukemia. According to some embodiments, the cancer is lymphoma. According to some embodiments, the cancer is myeloma. According to some embodiments, the cancer is tumors of the central nervous system. According to some embodiments, the cancer is breast cancer. According to some embodiments, the cancer is prostate cancer. According to some embodiments, the cancer is cervical cancer. According to some embodiments, the cancer is uterine cancer. According to some embodiments, the cancer is lung cancer. According to some embodiments, the cancer is ovarian cancer. According to some embodiments, the cancer is testicular cancer. According to some embodiments, the cancer is thyroid cancer. According to some embodiments, the cancer is astrocytoma. According to some embodiments, the cancer is glioma. According to some embodiments, the cancer is pancreatic cancer. According to some embodiments, the cancer is mesotheliomas. According to some embodiments, the cancer is gastric cancer. According to some embodiments, the cancer is liver cancer. According to some embodiments, the cancer is renal cancer. According to some embodiments, the cancer is nephroblastoma. According to some embodiments, the cancer is bladder cancer. According to some embodiments, the cancer is oesophageal cancer. According to some embodiments, the cancer is cancer of the larynx. According to some embodiments, the cancer is cancer of the parotid. According to some embodiments, the cancer is cancer of the biliary tract. According to some embodiments, the cancer is endometrial cancer. According to some embodiments, the cancer is adenocarcinomas. According to some embodiments, the cancer is small cell carcinomas. According to some embodiments, the cancer is neuroblastomas. According to some embodiments, the cancer is adrenocortical carcinomas. According to some embodiments, the cancer is epithelial carcinomas. According to some embodiments, the cancer is desmoid tumors. According to some embodiments, the cancer is desmoplastic small round cell tumors. According to some embodiments, the cancer is endocrine tumors. According to some embodiments, the cancer is Ewing sarcoma family tumors. According to some embodiments, the cancer is germ cell tumors. According to some embodiments, the cancer is hepatoblastomas. According to some embodiments, the cancer is hepatocellular carcinomas. According to some embodiments, the cancer is non-rhabdomyosarcome soft tissue sarcomas. According to some embodiments, the cancer is osteosarcomas. According to some embodiments, the cancer is peripheral primitive neuroectodermal tumors. According to some embodiments, the cancer is retinoblastomas. According to some embodiments, the cancer is rhabdomyosarcomas.

According to some embodiments, the chromosomal abnormality is a deletion, a duplication, or a combination thereof.

According to some embodiments, the subject is a human.

According to some embodiments, step b-3) comprises determining sequence read depth of one or more target segments and one or more non-target segments.

According to some embodiments, the reference sample is a biological sample from one or more subjects with cancer or a biological sample from one or more subjects not with cancer.

According to some embodiments, identifying the prognostic group of the subject as adverse includes the biological sample comprising one or more chromosomal abnormalities selected from the group consisting of t(6;9)(p23;q34); DEK-NUP214; t(v;11q23); KMT2A rearranged; t(9;22)(q34;q11); BCR-ABL1; inv(3)(q21q26) or t(3;3)(q21;q26); GATA2,MECOM(EVI1); del(5q); abn(17p); Complex karyotype; and monosomal karyotype.

According to some embodiments, identifying the prognostic group of the subject as adverse includes the biological sample comprising one or more chromosomal abnormalities selected from the group consisting of t(6;9)(p23;q34); DEK-NUP214; t(v;11q23); KMT2A rearranged; t(9;22)(q34;q11); BCR-ABL1; inv(3)(q21q26) or t(3;3)(q21;q26); GATA2,MECOM(EVI1); del(5q); abn(17p); Complex karyotype; and monosomal karyotype.

According to some embodiments, identifying the prognostic group of the subject as favorable includes the biological sample comprising one or more chromosomal abnormalities selected from the group consisting of t(8;21)(q22;q22); RUNX1-RUNX1T1; inv(16)(p13q22) or t(16;16)(p13;q22); CBFB-MYH11; and t(15;17) PML-RARA; and does not include one or more of chromosomal abnormalities selected from the group consisting of t(9;11)(p21;q23); MLLT3-KMT2A; t(6;9)(p23;q34); DEK-NUP214; t(v;11q23); KMT2A rearranged; t(9;22)(q34;q11); BCR-ABL1; inv(3)(q21q26) or t(3;3)(q21;q26); GATA2,MECOM(EVI1); del(5q); abn(17p); Complex karyotype; and monosomal karyotype.

According to some embodiments, identifying the prognostic group of the subject as favorable includes the biological sample comprising one or more chromosomal abnormalities selected from the group consisting of t(8;21)(q22;q22.1); RUNX1-RUNX1T1; inv(16)(p13.1q22) or t(16;16)(p13.1;q22); CBFB-MYH11; and t(15;17) PML-RARA; and does not include one or more of chromosomal abnormalities selected from the group consisting of t(9;11)(p21.3;q23.3); MLLT3-KMT2A; t(6;9)(p23;q34.1); DEK-NUP214; t(v;11q23.3); KMT2A rearranged; t(9;22)(q34.1;q11.2); BCR-ABL1; inv(3)(q21.3q26.2) or t(3;3)(q21.3;q26.2); GATA2,MECOM(EVI1); del(5q); abn(17p); Complex karyotype; and monosomal karyotype.

According to some embodiments, identifying the prognostic group of the subject as intermediate includes the biological sample comprising one or more chromosomal abnormalities selected from the group consisting of t(9;11)(p21;q23); MLLT3-KMT2A; and does not include t(8;21)(q22;q22); RUNX1-RUNX1T1; inv(16)(p13q22) or t(16;16)(p13;q22); CBFB-MYH11; t(15;17) PML-RARA; t(6;9)(p23;q34); DEK-NUP214; t(v;11q23); KMT2A rearranged; t(9;22)(q34;q11); BCR-ABL1; inv(3)(q21q26) or t(3;3)(q21;q26); GATA2,MECOM(EVI1); del(5q); abn(17p); complex karyotype; and monosomal karyotype.

According to some embodiments, identifying the prognostic group of the subject as intermediate includes the biological sample comprising one or more chromosomal abnormalities selected from the group consisting of t(9;11)(p21.3;q23.3); MLLT3-KMT2A; and does not include t(8;21)(q22;q22.1); RUNX1-RUNX1T1; inv(16)(p13.1q22) or t(16;16)(p13.1;q22); CBFB-MYH11; t(15;17) PML-RARA; t(6;9)(p23;q34.1); DEK-NUP214; t(v;11q23.3); KMT2A rearranged; t(9;22)(q34.1;q11.2); BCR-ABL1; inv(3)(q21.3q26.2) or t(3;3)(q21.3;q26.2); GATA2,MECOM(EVI1); del(5q); abn(17p); complex karyotype; and monosomal karyotype.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A depicts gain and loss of chromosomal regions in a sample with variant allele frequency of 13% (e.g., gene FLT3-TKD).

FIG. 1B depicts gain and loss of chromosomal regions in a sample with variant allele frequency of 8% (e.g., gene TP53), the lower limit to detect possible chromosomal structural abnormalities.

FIG. 2 depicts differences between cytogenetic and next generation sequencing (NGS) testing for patient nos. 2, 13, 21, and 24, as shown in Table 4.

FIG. 3 schematically depicts a network for implementing some aspects in accordance with some embodiments.

FIG. 4 schematically depicts a computing system for implementing some aspects in accordance with some embodiments.

### DETAILED DESCRIPTION OF THE INVENTION

As used herein and in the appended claims, the singular forms “a”, “an”, and “the” include plural reference unless the context clearly dictates otherwise. Thus, for example, reference to a “peptide” is a reference to one or more peptides and equivalents thereof known to those skilled in the art, and so forth.

The term “about” is used herein to mean within the typical ranges of tolerances in the art. For example, “about” can be understood as about 2 standard deviations from the mean. According to certain embodiments, about means +10%. According to certain embodiments, about means +5%. When about is present before a series of numbers or a range, it is understood that “about” can modify each of the numbers in the series or range.

The term “at least” prior to a number or series of numbers (e.g. “at least two”) is understood to include the number adjacent to the term “at least”, and all subsequent numbers or integers that could logically be included, as clear from context. When at least is present before a series of numbers or a range, it is understood that “at least” can modify each of the numbers in the series or range.

As used herein, “up to” as in “up to 10” is understood as up to and including 10, i.e., 0, 1, 2, 3, 4, 5, 6, 7, 8, 9, or 10.

Ranges provided herein are understood to include all individual integer values and all subranges within the ranges.

As used herein, the term “in combination with,” is not intended to imply that the therapy or the therapeutic agents must be administered at the same time and/or formulated for delivery together, although these methods of delivery are within the scope described herein. The therapeutic agents can be administered concurrently with, prior to, or subsequent to, one or more other additional therapies or therapeutic agents.

The term “active agent” refers to the ingredient, component or constituent of the compositions of the described invention responsible for the intended therapeutic effect.

The term “administer” as used herein means to give or to apply. The term “administering” as used herein includes in vivo administration, as well as administration directly to tissue ex vivo. “Administering” may be accomplished by any route as disclosed below.

As used herein, the term “base pair” or “bp” refers to a unit consisting of two nucleobases bound to each other by hydrogen bonds. Generally, the size of an organism's genome is measured in base pairs because DNA is typically double stranded. However, some viruses have single-stranded DNA or RNA genomes.

The term “biomarker” (or “biosignature”) as used herein refers to peptides, proteins, nucleic acids, antibodies, genes, metabolites, or any other substances used as indicators of a biologic state. It is a characteristic that is measured objectively and evaluated as a cellular or molecular indicator of normal biologic processes, pathogenic processes, or pharmacologic responses to a therapeutic intervention. In some embodiments, the biomarker(s) is the copy number of genes, which are altered in diseased samples compared to a reference sample. The term “indicator” as used herein refers to any substance, number or ratio derived from a series of observed facts that may reveal relative changes as a function of time; or a signal, sign, mark, note or symptom that is visible or evidence of the existence or presence thereof. Once a proposed biomarker has been validated, it may be used to diagnose disease risk, presence of disease in an individual, or to tailor treatments for the disease in an individual (e.g., choices of drug treatment or administration regimes). In some embodiments, the biomarker, meaning one or more genes, has/have an altered copy number, e.g., indicator, compared to a reference sample. In some embodiments, the indicator is that the copy number of one or more genes is decreased compared to a reference sample. In some embodiments, the indicator is that the copy number of one or more genes is increased compared to a reference sample.

As used herein, the term “cell-free DNA” and “cfDNA” interchangeably refer to DNA fragments that circulate in a subject's body (e.g., bloodstream) and originate from one or more healthy cells and/or from one or more cancer cells. These DNA molecules are found outside cells, in bodily fluids such as blood, whole blood, plasma, serum, urine, cerebrospinal fluid, fecal, saliva, sweat, sweat, tears, pleural fluid, pericardial fluid, or peritoneal fluid of a subject, and are believed to be fragments of genomic DNA expelled from healthy and/or cancerous cells, e.g., upon apoptosis and lysis of the cellular envelope.

The terms “chromosomal abnormality”, “chromosomal aberration” and “chromosomal alteration” are used herein interchangeably. They refer to a difference (i.e., a variation) in the number of chromosomes or to a difference (i.e., a modification) in the structural organization of one or more chromosomes as compared to chromosomal number and structural organization in a karyotypically normal individual. As used herein, these terms are also meant to encompass abnormalities taking place at the gene level. The presence of an abnormal number of (i.e., either too many or too few) chromosomes is called “aneuploidy”. Examples of aneuploidy are trisomy 21 and trisomy 13. Structural chromosomal abnormalities include: deletions (e.g., absence of one or more nucleotides normally present in a gene sequence, absence of an entire gene, or missing portion of a chromosome), additions (e.g., presence of one or more nucleotides usually absent in a gene sequence, presence of extra copies of a gene (also called duplication), or presence of an extra portion of a chromosome), rings, breaks and chromosomal rearrangements. Abnormalities that involve deletions or additions of chromosomal material alter the gene balance of an organism and if they disrupt or delete active genes, they generally lead to fetal death or to serious mental and physical defects. Structural rearrangements of chromosomes result from chromosome breakage caused by damage to DNA, errors in recombination, or crossing over the maternal and paternal ends of the separated double helix during meiosis or gamete cell division. Chromosomal rearrangements may be translocations or inversions. A translocation results from a process in which genetic material is transferred from one chromosome to another. A translocation is balanced when two chromosomes exchange pieces without loss of genetic material, while an unbalanced translocation occurs when chromosomes either gain or lose genetic material. Translocations may involve two chromosomes or only one chromosome. Inversions are produced by a process in which two breaks occur in a chromosome and the broken segment rotates 180°, resulting in the genes being rearranged in reverse order.

### Copy Number Variation

As used herein, the term “copy number variation” or “CNV” refers to a variation in the number of copies of a nucleic acid sequence present in a test sample in comparison with the copy number of the nucleic acid sequence present in a reference sample. In certain embodiments, the nucleic acid sequence is 1 kb or larger. In some cases, the nucleic acid sequence is a whole chromosome or significant portion thereof. A “copy number variant” refers to a sequence of nucleic acid in which copy-number differences are found by comparison of a nucleic acid sequence of interest in a test sample with an expected level of the nucleic acid sequence of interest. For example, the level of the nucleic acid sequence of interest in the test sample is compared to that present in a qualified sample. Copy number variants/variations include deletions, including microdeletions, insertions, including microinsertions, duplications, multiplications, and translocations. CNVs encompass chromosomal aneuploidies and partial aneuploidies.

De novo copy number variation can occur both in germline and in somatic cells and is generated most commonly through two mechanisms: (1) nonallelic homologous recombination (NAHR) and (2) nonhomologous end joining (NHEJ or microhomology-mediated end joining (MMEJ). NAHR results from incorrect pairing across large homologous regions resulting in gain or loss of intervening segments. Non homologous end joining commonly occurs during DNA repair or DNA recombination, wherein DNA ends are annealed without sequence homology.

As used herein, the term “germline variants” refers to genetic variants inherited from maternal and paternal DNA. Germline variants may be determined through a matched tumor-normal calling pipeline. A bioinformatics pipeline is a set of complex algorithms used to process sequence data in order to generate a list of variants or assemble a genome(s). As used herein, the term “somatic variants” refers to variants arising as a result of dysregulated cellular processes associated with neoplastic cells, e.g., a mutation. Somatic variants may be detected via subtraction from a matched normal sample.

In some embodiments, the methods described herein determine the copy number variation (CNV) of one or more selected or target genes or fragments of selected or target genes (e.g., bin-level sequence ratios, segment-level sequence ratios, and segment-level measures of dispersion) from sequencing a liquid biopsy sample and, optionally, one or more matching biological samples from the subject (e.g., a matched cancerous and/or matched non-cancerous sample from the subject) or a reference biological sample (e.g., a biological sample or set of biological samples from subjects with no diagnosed disease, e.g., cancer, or a biological sample or set of biological samples from subjects with diagnosed disease), e.g., as an output of a conventional bioinformatics tool (such as the CNVkit software package, which was developed at the University of California, San Francisco and is downloadable from the github depository). A description of the CNVkit software appears in the article “CNVkit: Genome-wide copy number detection and visualization from targeted sequencing” by Talevich et al., which is incorporated by reference herein in its entirety. [Talevich, E., Shain, A. H., Botton, T., & Bastian, B. C. (2014). CNVkit: Genome-wide copy number detection and visualization from targeted sequencing. PLOS Computational Biology 12(4):e1004873]

As used herein, the term “bin” or grammatical variations thereof refer to a subset of a larger grouping, e.g., a genome. Next generation sequencing calculations (e.g., CNVkit) are performed by first dividing the genome into small regions (bins), on which the calculations are actually performed. The genome is partitioned into bins with an expected equal number of mappable positions. In some embodiments, the bins divide the genome into small or large regions depending on target regions. For example, on-target and off-target regions may be partitioned into bins ranging as small as 100 by to as large as 1000 kb or more. On-target regions may be partitioned into smaller regions, while the off-target regions may be partitioned into larger regions. Copy number variation is determined by determining, the read depth for each region (e.g., on- or off-target regions).

Once the genome is partitioned and the bins are ordered, the sequence reads in each bin can be analyzed to determine “bin-level sequence ratios”. Bin-level sequence ratios can be determined by comparing corresponding bins between the sequence reads in each bin from the sample (e.g., DNA from cancerous cells) and from the reference sample (e.g., DNA from healthy cells). For example, each respective bin in the plurality of bins represents a corresponding region of a human reference genome, and each respective bin-level sequence ratio in the plurality of bin-level sequence ratios is determined from a sequencing of a plurality of cell-free nucleic acids in a first liquid biopsy sample of the test subject and one or more reference samples. For example, when the ratio between corresponding bins in the sample and reference is dose to 1, the copy numbers between the sample and reference are similar. When the ratio between corresponding bins in the sample and reference is greater or less than 1, the copy numbers between the sample and reference are dissimilar.

In some embodiments, copy number variation can be determined at the segment-level by determining the segment-level sequence ratios. Each respective segment in the plurality of segments represents a corresponding region of the human reference genome encompassing a subset of adjacent bins in the plurality of bins, and each respective segment-level sequence ratio in the plurality of segment-level sequence ratios is determined from a measure of central tendency of the plurality of bin-level sequence ratios corresponding to the subset of adjacent bins encompassed by the respective segment.

In some embodiments a plurality of segment-level measures of dispersion are determined for a biological sample and/or a reference sample, where each respective segment-level measure of dispersion in the plurality of segment-level measures of dispersion (i) corresponds to a respective segment in the plurality of segments and (ii) is determined using the plurality of bin-level sequence ratios corresponding to the subset of adjacent bins encompassed by the respective segment. As used herein, the term “dispersion”, “variability”, “scatter”, or “spread” refers to how similar a set of scores (e.g., bin-level sequence ratios or segment-level sequence ratios) are to each other. For example, the more similar the scores are (e.g., the bin-level sequence ratio is close to 1) the lower the measure of dispersion will be and the more squeezed a distribution is in a dataset. For example, the less similar the scores are (e.g., the bin-level sequence ratio is greater or less than 1) to each other, the higher the measure of dispersion will be and the more stretched a distribution is in a dataset.

In some embodiments, the method includes obtaining a dataset including cell-free DNA sequencing data, and determining the copy number variation of one or more genes or gene segments. As used herein, the term “gene segment” refers to a region of the genome, more specifically a region of a gene. For instance, sequence reads are aligned and sequence read depth is determined compared to internal standard number of sequence reads (e.g., sequence reads of a genomic segment directly upstream or downstream of the genomic region of interest). As used herein, the term “read depth” or “depth of coverage” refers to the number of reads of a given nucleotide in an experiment. Most NGS protocols start with a random fragmentation of the genome into short random fragments. These fragments are then sequenced and aligned. This alignment creates a longer contiguous sequence, by tiling of the short sequences. In some embodiments, the CNV is determined by applying a copy number segmentation algorithm to the sequencing data.

In some embodiments, sequence reads are obtained from the sequencing dataset and aligned to a reference sample, generating a plurality of aligned reads and further processed using a copy number variation algorithm, which may be implemented in software (e.g., CNVkit). For instance, the copy number variation algorithm implemented in software (e.g., CNVkit) is used for genomic region binning (e.g., bin values, e.g., defining segment size while partitioning the genome), coverage calculation, bias correction, normalization to a reference pool, segmentation, and/or visualization. Bin values are used to determine bin-level sequence ratios between various genomic segments.

Next Generation Sequencing (NGS) has the potential to introduce several biases into the dataset. Bias in sequencing data can result from chromatin structure, enzymatic cleavage, nucleic acid isolation, PCR amplification, and read mapping effects. Both mechanical and enzymatic methods of fragmenting the genome can result in uneven-sized fragments. For example, heterochromatin (e.g., gene segments without coding regions) is more resistant to shearing by sonication than euchromatin (e.g., gene segments under active transcription) because of the more open configuration of euchromatin making it more vulnerable to shearing. Enzymatic digestion can introduce biases depending on the cleavage enzyme used. For example, MNase has a preference of digesting AT rich regions. Nucleic acid isolation can be incomplete when some DNA is bound by various polypeptides. PCR amplification can introduce bias because the PCR cycle can prefer some segments over others depending on denature and annealing temperatures, polymerase and buffer. Lastly, the sample data set is mapped on to a reference sample, which can introduce a bias toward the specific sequence of the reference sample. See Meyer CA, Liu XS. Identifying and mitigating bias in next-generation sequencing methods for chromatin biology. [Nat Rev Genet. 2014; 15(10:709-721.]

Bias correction for varied GC content can be determined by fitting a rolling median, then subtracting from the original read depths in a sample to yield corrected estimates.

As used herein, the term “normalization” in next-generation sequencing (NGS) is the process of equalizing the concentration of DNA libraries for multiplexing (e.g., annealing individual barcode sequences to individual fragments.

For instance, CNVkit, uses both on-target reads (e.g., genomic segment of interest) and off-target-reads (e.g., genomic region included in the sequencing dataset not specifically sequenced) to calculate loge copy ratios across the genome or a select segments of the genome. On- and off-target locations are separately determined and used to calculate the mean read depth within each segment of interest. On- and off-target depth reads are combined, normalized to a reference sample, corrected for systemic biases to result in final loge copy ratios. (See Talevich E, Shain A H, Botton T, Bastian B C. CNVkit: Genome-Wide Copy Number Detection and Visualization from Targeted DNA Sequencing. PLoS Comput Biol. 2016 Apr. 21; 12(4):e1004873. doi: 10.1371/journal.pcbi.1004873).

As used herein, “off-target intervals” refers to nonspecifically captured off-target reads.

In some embodiments, the copy number variation algorithm (e.g., CNVkit) corrects biases, e.g., genomic GC content and sequence repeats. Genomic GC rich regions are less accessible to hybridization and are less amenable to amplification during sample preparation. For instance, CNVkit applies a rolling median correction to GC values in both on- and off-target bins. Id.

The term “aneuploidy” herein refers to an imbalance of genetic material caused by a loss or gain of a whole chromosome, or part of a chromosome.

The terms “chromosomal aneuploidy” and “complete chromosomal aneuploidy” herein refer to an imbalance of genetic material caused by a loss or gain of a whole chromosome, and includes germline aneuploidy and mosaic aneuploidy.

The terms “partial aneuploidy” and “partial chromosomal aneuploidy” herein are used interchangeably to refer to an imbalance of genetic material caused by a loss or gain of part of a chromosome, e.g., partial monosomy and partial trisomy, and encompass imbalances resulting from translocations, deletions and insertions.

As used herein, the term “derived from” refers to any method for receiving, obtaining, or modifying something from a source of origin.

The terms “decrease” or “reduce” and their various grammatical forms are used herein to refer to a diminution, a reduction, an attenuation or abatement of the degree, intensity, extent, size, amount, density or number of occurrences, events or characteristics. As used herein, the term “increase” and its various grammatical forms refers to becoming or making greater in size, amount, intensity, or degree, such as a n increase in the number of occurrences, events or characteristics.

The term “effective amount,” is used herein to include the amount of an agent that, when administered to a patient for treating a subject having a disease, e.g., cancer, is sufficient to effect treatment of the disease (e.g., by diminishing, ameliorating or maintaining the existing disease or one or more symptoms of disease or its related comorbidities). The “effective amount” may vary depending on the agent, how it is administered, the disease and its severity and the history, age, weight, family history, genetic makeup, stage of pathological processes, the types of preceding or concomitant treatments, if any, and other individual characteristics of the patient to be treated. An effective amount includes an amount that results in a clinically relevant change or stabilization, as appropriate, of an indicator of a disease or condition. The term includes prophylactic or preventative amounts of the compositions of the described invention. In prophylactic or preventative applications of the described invention, pharmaceutical compositions or medicaments are administered to a patient susceptible to, or otherwise at risk of, a disease, disorder or condition in an amount sufficient to eliminate or reduce the risk, lessen the severity, or delay the onset of the disease, disorder or condition, including biochemical, histologic and/or behavioral symptoms of the disease, disorder or condition, its complications, and intermediate pathological phenotypes presenting during development of the disease, disorder or condition. It is generally preferred that a maximum dose be used, that is, the highest safe dose according to some medical judgment. The terms “dose” and “dosage” are used interchangeably herein.

As used herein, the term “exome sequencing” or grammatically variations thereof refers to sequencing of all or select regions of protein-coding regions of genes, e.g., exons. Exome DNA is enriched by isolation of exon DNA by one or more exon-specific markers, e.g., histone modifications, e.g., H3K9ac and H3K4me3.

The term “expression” as used herein generally refers to the action of a gene in the production of a protein or phenotype. More specifically, it refers to the process by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell. Expression may also refer to the post-translational modification of a polypeptide or protein.

As used herein, the terms “expression level,” “abundance level,” or simply “abundance” refers to an amount of a gene product, (an RNA species, e.g., mRNA or miRNA, or protein molecule) transcribed or translated by a cell, or an average amount of a gene product transcribed or translated across multiple cells. When referring to mRNA or protein expression, the term generally refers to the amount of any RNA or protein species corresponding to a particular genomic locus, e.g., a particular gene. However, in some embodiments, an expression level can refer to the amount of a particular isoform of an mRNA or protein corresponding to a particular gene that gives rise to multiple mRNA or protein isoforms.

The term “gene” as used herein refers to a region of DNA that controls a discrete hereditary characteristic, usually corresponding to a single protein or RNA. This definition includes the entire functional unit, encompassing coding DNA sequences, noncoding regulatory DNA sequences, and introns.

As used herein, the term “gene fusion” refers to the product of large scale chromosomal aberrations resulting in the creation of a chimeric protein. These expressed products can be non-functional, or they can be highly over or underactive. This can cause deleterious effects in cancer such as hyper-proliferative or anti-apoptotic phenotypes.

As used herein, the terms “genomic alteration,” “mutation,” and “variant” refer to a detectable change in the genetic material of one or more cells. A genomic alteration, mutation, or variant can refer to various type of changes in the genetic material of a cell, including changes in the primary genome sequence at single or multiple nucleotide positions, e.g., a single nucleotide variant (SNV), a multi-nucleotide variant (MNV), an indel (e.g., an insertion or deletion of nucleotides), a DNA rearrangement (e.g., an inversion or translocation of a portion of a chromosome or chromosomes), a variation in the copy number of a locus (e.g., an exon, gene, or a large span of a chromosome) (e.g., copy number variation “CNV”), a partial or complete change in the ploidy of the cell, as well as in changes in the epigenetic information of a genome, such as altered DNA methylation patterns. In some embodiments, a mutation is a change in the genetic information of the cell relative to a particular reference genome, or one or more ‘normal’ alleles found in the population of the species of the subject. For instance, mutations can be found in both germline cells (e.g., non-cancerous, ‘normal’ cells) of a subject and in abnormal cells (e.g., pre-cancerous or cancerous cells) of the subject. As such, a mutation in a germline of the subject (e.g., which is found in substantially all ‘normal cells’ in the subject) is identified relative to a reference genome for the species of the subject. However, many loci of a reference genome of a species are associated with several variant alleles that are significantly represented in the population of the subject and are not associated with a diseased state, e.g., such that they would not be considered ‘disease-causing.’ By contrast, in some embodiments, a mutation in a cancerous cell of a subject can be identified relative to either a reference genome of the subject or to the subject's own germline genome. In certain instances, identification of both types of variants can be informative. For instance, in some instances, a mutation that is present in both the cancer genome of the subject and the germline of the subject is informative for precision oncology when the mutation is a so-called ‘driver mutation,’ which contributes to the initiation and/or development of a cancer. However, in other instances, a mutation that is present in both the cancer genome of the subject and the germline of the subject is not informative for precision oncology, e.g., when the mutation is a so-called ‘passenger mutation,’ which does not contribute to the initiation and/or development of the cancer. Likewise, in some instances, a mutation that is present in the cancer genome of the subject but not the germline of the subject is informative for precision oncology, e.g., where the mutation is a driver mutation and/or the mutation facilitates a therapeutic approach, e.g., by differentiating cancer cells from normal cells in a therapeutically actionable way. However, in some instances, a mutation that is present in the cancer genome but not the germline of a subject is not informative for precision oncology, e.g., where the mutation is a passenger mutation and/or where the mutation fails to differentiate the cancer cell from a germline cell in a therapeutically actionable way.

As used herein, the term “single nucleotide variant” or “SNV” refers to a substitution of one nucleotide to a different nucleotide at a position (e.g., site) of a nucleotide sequence, e.g., a sequence read from an individual. A substitution from a first nucleobase X to a second nucleobase Y may be denoted as “X>Y.” For example, a cytosine to thymine SNV may be denoted as “C>T.”

As used herein, the term “insertions and deletions” or “indels” refers to a variant resulting from the gain or loss of DNA base pairs within an analyzed region.

The term “inhibitor” as used herein refers to a molecule that reduces the amount or rate of a process, stops the process entirely, or that decreases, limits, or blocks the action or function thereof. Enzyme inhibitors are molecules that bind to enzymes thereby decreasing enzyme activity. Inhibitors may be evaluated by their specificity and potency.

As used herein, the term “karyotype” refers to the particular chromosome complement of an individual or a related group of individuals, as defined by the number and morphology of the chromosomes usually in mitotic metaphase. More specifically, a karyotype includes such information as total chromosome number, copy number of individual chromosome types (e.g., the number of copies of chromosome Y) and chromosomal morphology (e.g., length, centromeric index, connectedness and the like). Examination of a karyotype allows detection and identification of chromosomal abnormalities (e.g., extra, missing, or broken chromosomes). Since certain diseases and conditions are associated with characteristic chromosomal abnormalities, analysis of a karyotype allows diagnosis of these diseases and conditions. In some embodiments, karyotype nomenclature is described in Table 1.

TABLE 1

| Symbol | Description |
|---|---|
| 1-22 | Autosome |
| X, Y | Sex chromosome |
| (+) or (−) | When placed before an autosomal number, this indicates that |
| | the chromosome is an extra or is missing |
| add | added material of unknown origin, typically resulting in a loss |
| | of material distal to breakpoint |
| c | constitutional |
| cp | composite (clonal, but variable across cells) |
| cen | Centromere |
| inv | Inversion |
| dic | Dicentric |
| dmin | double minute chromosome |
| dn | de novo (not inherited) |
| i | Isochromosome (composed of two identical chromosome |
| | arms) |
| idic | isodicentric chromosome (isochromosome w/two centromeres) |
| p | Short arm of the chromosome |
| q | Long arm of the chromosome |
| r | Ring chromosome |
| t | Translocation |
| del | Deletion |
| ins | Insertion |
| dup | Duplication |
| mar | marker chromosome, unknown origin |
| mat | maternal origin |
| mos | mosaic (multiple cell lines/clones present) |
| pat | paternal origin |
| rob | Robertsonian translocation, a whole arm translocation between |
| | acrocentric chromosomes |
| sl | stemline (used with clonal |
| sdl | sideline (used with clonal evolution) |
| ter | Terminal |
| der | Derivative; derivative chromosome, due to structural |
| | rearrangement(s) |
| ? | designates uncertainty (used in place of, or in front of a |
| | finding) |
| / | separates clones (for mosaic karyotypes) |
| // | separates clones (for chimeric karyotypes) |
| [ ] | indicate number of cells (for mosaic or chimeric karyotypes) |
| : | Break |
| :: | Break and join |
| → | From to |

As used herein, the term “complex karyotype” refers to the presence of more than or equal to 3 or more than or equal to 5 chromosomal aberrations. Structurally complex karyotype is defined by at least 3 chromosomal aberrations, including at least one structural aberration, excluding those with clonal evolution of monosomy 7. See Gohring, G. et al. “Complex karyotype newly defined: the strongest prognostic factor in advanced childhood myelodysplastic syndrome,” Blood (2010): 116 (19): 3766-69.

As used herein, the term “monosomal karyotype” refers to the presence of at least 2 autosomal monosomies or a single autosomal monosomy associated with at least one structural abnormality.

As used herein, the term “liquid biopsy” sample refers to any sample taken from a subject, which can reflect a biological state associated with the subject, and that includes cell-free DNA. Examples of sources of liquid biopsy samples include, but are not limited to, blood, whole blood, plasma, serum, urine, cerebrospinal fluid, fecal, saliva, sweat, tears, pleural fluid, pericardial fluid, or peritoneal fluid of the subject. A liquid biopsy sample can include any tissue or material derived from a living or dead subject. A liquid biopsy sample can be a cell-free sample. A liquid biopsy sample can comprise a nucleic acid (e.g., DNA or RNA) or a fragment thereof. The term “nucleic acid” can refer to deoxyribonucleic acid (DNA), ribonucleic acid (RNA) or any hybrid or fragment thereof.

As used herein, the term “locus” refers to a position (e.g., a site) within a genome, e.g., on a particular chromosome. In some embodiments, a locus refers to a single nucleotide position, on a particular chromosome, within a genome. In some embodiments, a locus refers to a group of nucleotide positions within a genome. In some instances, a locus is defined by a mutation (e.g., substitution, insertion, deletion, inversion, or translocation) of consecutive nucleotides within a cancer genome. In some instances, a locus is defined by a gene, a sub-genic structure (e.g., a regulatory element, exon, intron, or combination thereof), or a predefined span of a chromosome. Because normal mammalian cells have diploid genomes, a normal mammalian genome (e.g., a human genome) will generally have two copies of every locus in the genome, or at least two copies of every locus located on the autosomal chromosomes, e.g., one copy on the maternal autosomal chromosome and one copy on the paternal autosomal chromosome. As used herein, the term “allele” refers to a particular sequence of one or more nucleotides at a chromosomal locus. In a haploid organism, the subject has one allele at every chromosomal locus. In a diploid organism, the subject has two alleles at every chromosomal locus.

As used herein, the term “Next Generation Sequencing” or “NGS” refers to a method of parallel sequencing. For instance, a nucleic acid (e.g., DNA) sample is obtained and prepared into a library (meaning a collection of nucleic acid fragments from the sample). The library is prepared by fragmenting the DNA or RNA sample. Fragmentation can be performed by physical (e.g., sheared by acoustics, nebulization, centrifugal force, needles, or hydrodynamics) or enzymatic (e.g., site-specific or non-specific nucleases) methods. In some embodiments, the fragments are about 100 bp, about 150 bp, about 200 bp, about 250 bp, about 300 bp, about 350 bp, about 400 bp, about 450 bp, about 500 bp, about 550 bp, or about 600 bp in length. The DNA or RNA samples are repaired at the ends (e.g., blunt-ended) and then A-tailed (e.g., an adenosine is added to the 3′ end resulting in an overhang). Adapters are ligated to each end. Adapters include sequences, such as barcodes, restriction sites, and primer sequences.

As used herein, the term “coverage” in reference to NGS refers to the average number of reads that align to, or “cover” known reference basis. The sequencing coverage level determines whether variant discovery can be made with a certain degree of confidence at particular base positions. Coverage equals read count multiplied by the read length and divided by the total genome size. At higher the level of coverage, each base is covered by a greater number of aligned sequence reads, and mutations at the base level compared to a reference sample can be determined.

As used herein, the term “reference allele” refers to the sequence of one or more nucleotides at a chromosomal locus that is either the predominant allele represented at that chromosomal locus within the population of the species (e.g., the “wild-type” sequence), or an allele that is predefined within a reference genome for the species.

As used herein, the term “variant allele” refers to a sequence of one or more nucleotides at a chromosomal locus that is either not the predominant allele represented at that chromosomal locus within the population of the species (e.g., not the “wild-type” sequence), or not an allele that is predefined within a reference genome for the species. As used herein, the term “variant allele fraction,” “VAF,” “allelic fraction,” or “AF” refers to the number of times a variant or mutant allele was observed (e.g., a number of reads supporting a candidate variant allele) divided by the total number of times the position was sequenced (e.g., a total number of reads covering a candidate locus).

As used herein, the term “loss of heterozygosity” refers to the loss of one copy of a segment (e.g., including part or all of one or more genes) of the genome of a diploid subject (e.g., a human) or loss of one copy of a sequence encoding a functional gene product in the genome of the diploid subject, in a tissue, e.g., a cancerous tissue, of the subject. As used herein, when referring to a metric representing loss of heterozygosity across the entire genome of the subject, loss of heterozygosity is caused by the loss of one copy of various segments in the genome of the subject. Loss of heterozygosity across the entire genome may be estimated without sequencing the entire genome of a subject, and such methods for such estimations based on gene panel targeting-based sequencing methodologies are described in the art. Accordingly, in some embodiments, a metric representing loss of heterozygosity across the entire genome of a tissue of a subject is represented as a single value, e.g., a percentage or fraction of the genome. In some cases a tumor is composed of various sub-clonal populations, each of which may have a different degree of loss of heterozygosity across their respective genomes. Accordingly, in some embodiments, loss of heterozygosity across the entire genome of a cancerous tissue refers to an average loss of heterozygosity across a heterogeneous tumor population. As used herein, when referring to a metric for loss of heterozygosity in a particular gene, e.g., a DNA repair protein such as a protein involved in the homologous DNA recombination pathway (e.g., BRCA1 or BRCA2), loss of heterozygosity refers to complete or partial loss of one copy of the gene encoding the protein in the genome of the tissue and/or a mutation in one copy of the gene that prevents translation of a full-length gene product, e.g., a frameshift or truncating (creating a premature stop codon in the gene) mutation in the gene of interest. In some cases a tumor is composed of various sub-clonal populations, each of which may have a different mutational status in a gene of interest. Accordingly, in some embodiments, loss of heterozygosity for a particular gene of interest is represented by an average value for loss of heterozygosity for the gene across all sequenced sub-clonal populations of the cancerous tissue. In other embodiments, loss of heterozygosity for a particular gene of interest is represented by a count of the number of unique incidences of loss of heterozygosity in the gene of interest across all sequenced sub-clonal populations of the cancerous tissue (e.g., the number of unique frame-shift and/or truncating mutations in the gene identified in the sequencing data).

The term “messenger RNA” (“mRNA”) as used herein refers to a coding RNA that functions in protein translation. The term “RNA molecule” or “ribonucleic acid molecule,” as used herein, refers to a linear, single-stranded polymer composed of ribose nucleotides that is synthesized by transcription of DNA or by copying of RNA. It encompasses not only RNA molecules as expressed or found in nature, but also analogs and derivatives of RNA comprising one or more ribonucleotide/ribonucleoside analogs or derivatives as described herein or as known in the art. Strictly speaking, a “ribonucleoside” includes a nucleoside base and a ribose sugar, and a “ribonucleotide” is a ribonucleoside with one, two or three phosphate moieties.

The term “microRNA” (or “miRNA” or “miR”) as used herein refers to a class of small, 18- to 28-nucleotide-long, noncoding RNA molecules.

The term “pharmaceutical composition” is used herein to refer to a composition that is employed to prevent, reduce in intensity, cure or otherwise treat a target condition or disease. The terms “formulation” and “composition” are used interchangeably herein to refer to a product of the described invention that comprises all active and inert ingredients.

The term “pharmaceutically acceptable,” is used to refer to the carrier, diluent or excipient being compatible with the other ingredients of the formulation or composition and not deleterious to the recipient thereof. The carrier must be of sufficiently high purity and of sufficiently low toxicity to render it suitable for administration to the subject being treated. The carrier further should maintain the stability and bioavailability of an active agent. For example, the term “pharmaceutically acceptable” can mean approved by a regulatory agency of the Federal or a state government or listed in the U.S. Pharmacopeia or other generally recognized pharmacopeia for use in animals, and more particularly in humans.

As used herein, each of the terms “peptide,” “polypeptide” and “protein” refer to two or more amino acids covalently linked by an amide bond or non-amide equivalent. A peptide generally is considered an amino acid polymer of 40 or fewer amino acids. A polypeptide generally is considered an amino acid polymer containing more than 40 amino acids but less than 100 amino acids. A protein generally is considered an amino acid polymer with a defined sequence that is greater than 100 amino acids in size. The amino acid polymers of the present disclosure can be of any length. For example, they can have from about two to about 100 or more residues, such as, 5 to 12, 12 to 15, 15 to 18, 18 to 25, 25 to 50, 50 to 75, 75 to 100, 100 to 200, or more in length. The amino acid sequence can include L- and D-isomers, and combinations of L- and D-isomers. The amino acid sequence also can include modifications typically associated with post-translational processing of proteins, for example, cyclization (e.g., disulfide or amide bond), phosphorylation, glycosylation, carboxylation, ubiquitination, myristylation, or lipidation.

The term “pharmaceutically acceptable,” is used to refer to the carrier, diluent or excipient being compatible with the other ingredients of the formulation or composition and not deleterious to the recipient thereof. For example, the term “pharmaceutically acceptable” can mean approved by a regulatory agency of the Federal or a state government or listed in the U.S. Pharmacopeia or other generally recognized pharmacopeia for use in animals, and more particularly in humans.

The term “progression” is used herein to refer to the course of a disease as it becomes worse.

As used herein, the term “prognostic group” refers to a category of disease progression as compared to a reference sample (e.g., favorable, intermediate, adverse (poor), or complex prognosis). In some embodiments, the category of disease progression as compared to a reference sample is “favorable” prognosis. In some embodiments, favorable prognosis may include chromosome aberrations at t(8;21)(q22;q22); RUNX1-RUNX1T1; inv(16)(p13q22) or t(16;16)(p13;q22); CBFB-MYH11; and/or t(15;17) PML-RARA. In some embodiments, favorable prognosis may include chromosome aberrations at t(8;21)(q22;q22.1); RUNX1-RUNX1T1; inv(16)(p13.1q22) or t(16;16)(p13.1;q22); CBFB-MYH11; and/or t(15;17) PML-RARA. In some embodiments, the category of disease progression as compared to a reference sample is “intermediate” prognosis. In some embodiments, intermediate prognosis may include chromosome aberrations at t(9;11)(p21;q23); MLLT3-KMT2A and/or cytogenetic abnormalities not classified as favorable or adverse. In some embodiments, intermediate prognosis may include chromosome aberrations at t(9;11)(p21.3;q23.3); MLLT3-KMT2A and/or cytogenetic abnormalities not classified as favorable or adverse. In some embodiments, the category of disease progression as compared to a reference sample is “adverse (poor)” prognosis. In some embodiments, adverse (poor) prognosis may include chromosome aberrations at t(6;9)(p23;q34); DEK-NUP214; t(v;11q23); KMT2A rearranged; t(9;22)(q34;q11); BCR-ABL1; inv(3)(q21q26) or t(3;3)(q21;q26); GATA2,MECOM(EVI1); del(5q); abn(17p); complex karyotype; and/or monosomal karyotype. In some embodiments, adverse (poor) prognosis may include chromosome aberrations at t(6;9)(p23;q34.1); DEK-NUP214; t(v;11q23.3); KMT2A rearranged; t(9;22)(q34.1;q11.2); BCR-ABL1; inv(3)(q21.3q26.2) or t(3;3)(q21.3;q26.2); GATA2,MECOM(EVI1); del(5q); abn(17p); complex karyotype; and/or monosomal karyotype. In some embodiments, the category of disease progression as compared to a reference sample is “complex” prognosis, wherein the complex prognostic group is a subgroup of the adverse prognostic group.

Examples of common chromosomal aberrations in cancer are listed in Table 2.

TABLE 2

| Neoplasm | Translocation |
|---|---|
| Chronic myelogenous leukemia | t(9; 22)(q34; q11) |
| Acute lymphocytic leukemia | t(9; 22)(q34; q11) |
| Acute lymphocytic leukemia | t(4; 11)(q21; q23) |
| Acute promyelocytic leukemia | t(15; 17)(q22; q21) |
| Acute myeloid leukemia | t(8; 21)(q22; q22) |
| Acute myeloid leukemia | inv(16)(p13.3q22) or |
| | t(16; 16)(p13; q22) |
| Burkitt lymphoma | t(8; 14)(q24; q32) |
| | t(8; 22)(q24; q11) |
| | t(2; 8)(q11; q24) |

The term “quantitative PCR” (or “qPCR”), also called “real time-PCR” or “quantitative real-time PCR” refers to a polymerase chain reaction-based technique that couples amplification of a target DNA sequence with quantification of the concentration of that DNA species in the reaction.

As used herein, the term “sequence reads” or “reads” refers to nucleotide sequences produced by any nucleic acid sequencing process described herein or known in the art. Reads can be generated from one end of nucleic acid fragments (“single-end reads”) or from both ends of nucleic acid fragments (e.g., paired-end reads, double-end reads). The length of the sequence read is often associated with the particular sequencing technology. High-throughput methods, for example, provide sequence reads that can vary in size from tens to hundreds of base pairs (bp). In some embodiments, the sequence reads are of a mean, median or average length of about 15 bp to 900 bp long (e.g., about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, or about 500 bp. In some embodiments, the sequence reads are of a mean, median or average length of about 1000 bp, 2000 bp, 5000 bp, 10,000 bp, or 50,000 bp or more. Nanopore® sequencing methods and associated devices provided by Oxford Nanopore Technology PLC of Oxford, UK, for example, can provide sequence reads that can vary in size from tens to hundreds to thousands of base pairs. Illumina® parallel sequencing methods and associated devices provided by Illumina Inc. of San Diego, CA, for example, can provide sequence reads that do not vary as much, for example, most of the sequence reads can be smaller than 200 bp. A sequence read (or sequencing read) can refer to sequence information corresponding to a nucleic acid molecule (e.g., a string of nucleotides). For example, a sequence read can correspond to a string of nucleotides (e.g., about 20 to about 150) from part of a nucleic acid fragment, can correspond to a string of nucleotides at one or both ends of a nucleic acid fragment, or can correspond to nucleotides of the entire nucleic acid fragment. A sequence read can be obtained in a variety of ways, e.g., using sequencing techniques or using probes, e.g., in hybridization arrays or capture probes, or amplification techniques, such as the polymerase chain reaction (PCR) or linear amplification using a single primer or isothermal amplification.

The term “signature” as used herein refers to a specific and complex combination of biomarkers that reflect a biological state. In some embodiments, the signature comprises a copy number variation indicative of disease or progression of a disease, e.g., cancer or the progression of the cancer.

The terms “subject”, “animal,” and “patient,” are used interchangeably to refer, for example, and without limitation, to humans and non-human vertebrates such as wild, domestic and farm animals. According to some embodiments, the terms “animal,” “patient,” and “subject” may refer to humans. According to some embodiments, the terms “animal,” “patient,” and “subject” may refer to non-human mammals. According to some embodiments, the terms “animal,” “patient,” and “subject” may refer to any or combination of: dogs, cats, pigs, cows, horses, goats, sheep or other domesticated non-human mammals.

The term a “subject in need” of treatment for a particular condition can be a subject having that condition, diagnosed as having that condition, or at risk of developing that condition.

The term “therapeutic agent” as used herein refers to a drug, molecule, composition or other substance that provides a therapeutic effect. The term “active” as used herein refers to the ingredient, component or constituent of the compositions of the present invention responsible for the intended therapeutic effect. The terms “therapeutic agent” and “active agent” are used interchangeably.

The term “therapeutic component” as used herein refers to a therapeutically effective dosage (i.e., dose and frequency of administration) that eliminates, reduces, or prevents the progression of a particular disease manifestation in a percentage of a population. An example of a commonly used therapeutic component is the ED50 which describes the dose in a particular dosage that is therapeutically effective for a particular disease manifestation in 50% of a population.

The term “therapeutic effect” as used herein refers to a consequence of treatment, the results of which are judged to be desirable and beneficial. A therapeutic effect may include, directly or indirectly, the arrest, reduction, or elimination of a disease manifestation. A therapeutic effect may also include, directly or indirectly, the arrest reduction or elimination of the progression of a disease manifestation.

The term “treat” or “treating” includes abrogating, substantially inhibiting, slowing or reversing the progression of a disease, condition or disorder, substantially ameliorating clinical or esthetical symptoms of a condition, substantially preventing the appearance of clinical or esthetical symptoms of a disease, condition, or disorder, and protecting from harmful or annoying symptoms. The term “treat” or “treating” as used herein further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously symptomatic for the disorder(s). Treatment includes eliciting a clinically significant response without excessive levels of side effects. Treatment also includes prolonging survival as compared to expected survival if not receiving treatment.

### Embodiments

### Methods

According to one aspect, the present disclosure provides a method for dividing subjects diagnosed with cancer or suspected of having cancer into different prognostic groups, the method comprising:
a) providing a biological sample from the subject;
b) determining copy number of one or more target genes or fragments of the target genes compared to a reference sample by: 1

1) preparing a cell-free DNA (cfDNA) sample from the biological sample of step a); 1

2) preparing a sequencing library from the cfDNA from the biological sample, wherein preparing the library comprises consecutive steps of fragmenting the cfDNA, end-repairing, dA-tailing and adaptor ligating the cfDNA fragments;
3) sequencing the adaptor-ligated cfDNA fragments of the one or more target genes or fragments thereof from the biological sample;
4) determining copy number of the one or more target genes or fragments thereof from the sequences of the adaptor-ligated cfDNA fragments from the biological sample;
5) comparing the copy number of the one or more target genes or cfDNA fragments thereof for the biological sample with that of a reference sample, wherein the comparison can determine one or more chromosomal abnormalities in the biological sample; and
   c) identifying the prognostic group of the subject based on step b), wherein the presence of one or more chromosomal abnormalities in the one or more target genes or fragments thereof for the biological sample when compared to the reference sample indicates a subject having cancer with a poor, intermediate, or complex prognosis; and
   d) qualifying the subject for chemotherapy or immunotherapy where the results in (c) indicate an intermediate or complex prognosis.

According to some embodiments, the sample is a tissue biopsy of the cancer or a liquid biopsy. In some embodiments, the sample is blood, plasma, serum, urine, stool, saliva, tissue, or bodily fluid. In some embodiments, the sample is a plasma sample derived from peripheral blood that comprises a mixture of cfDNA derived from normal and cancerous cells.

According to some embodiments, the one or more target genes or fragments thereof are selected from Table 3. Table 3 includes 275 genes that are selected according to cancer association with various cancers.

According to some embodiments, the target gene or fragments thereof are exons. Exons comprise protein-coding regions of the genome. Specific amplification of exons can be performed selecting exon-specific nucleic acid markers, such as histone modifications, e.g., H3K9ac and H3K4me3.

According to some embodiments, the prepared sequence library is sequenced by next generation sequencing (NGS). In some embodiments, step b-4) comprises determining sequence read depth of one or more target segments and one or more non-target segments.

According to some embodiments, the qualifying the subject for chemotherapy or immunotherapy comprises one or more of: displaying on a graphical user interface an identification of the subject as a candidate for chemotherapy or immunotherapy; storing data identifying the subject as a candidate for chemotherapy or immunotherapy; sending an electronic communication including an identification of the subject as a candidate for chemotherapy or immunotherapy; displaying on a graphical user interface a recommendation of chemotherapy or immunotherapy for the subject; storing data including a recommendation of immunotherapy or chemotherapy for the subject; and sending an electronic communication including a recommendation of immunotherapy or chemotherapy for the subject.

According to some embodiments, the identifying the prognostic group of the subject as adverse includes the biological sample comprising one or more chromosomal abnormalities selected from the group consisting of t(6;9)(p23;q34); DEK-NUP214; t(v;11q23); KMT2A rearranged; t(9;22)(q34;q11); BCR-ABL1; inv(3)(q21q26) or t(3;3)(q21;q26); GATA2,MECOM(EVI1); del(5q); abn(17p); complex karyotype; and monosomal karyotype.

According to some embodiments, the identifying the prognostic group of the subject as adverse includes the biological sample comprising one or more chromosomal abnormalities selected from the group consisting of t(6;9)(p23;q34.1); DEK-NUP214; t(v;11q23.3); KMT2A rearranged; t(9;22)(q34.1;q11.2); BCR-ABL1; inv(3)(q21.3q26.2) or t(3;3)(q21.3;q26.2); GATA2,MECOM(EVI1); del(5q); abn(17p); complex karyotype; and monosomal karyotype.

According to some embodiments, the identifying the prognostic group of the subject as favorable includes the biological sample comprising one or more chromosomal abnormalities selected from the group consisting of t(8;21)(q22;q22); RUNX1-RUNX1T1; inv(16)(p13q22) or t(16;16)(p13;q22); CBFB-MYH11; and t(15;17) PML-RARA; and does not include one or more chromosomal abnormalities selected from the group consisting of t(9;11)(p21;q23); MLLT3-KMT2A; t(6;9)(p23;q34); DEK-NUP214; t(v;11q23); KMT2A rearranged; t(9;22)(q34;q11); BCR-ABL1; inv(3)(q21q26) or t(3;3)(q21;q26); GATA2,MECOM(EVI1); del(5q); abn(17p); complex karyotype; and monosomal karyotype.

According to some embodiments, the identifying the prognostic group of the subject as favorable includes the biological sample comprising one or more chromosomal abnormalities selected from the group consisting of t(8;21)(q22;q22.1); RUNX1-RUNX1T1; inv(16)(p13.1q22) or t(16;16)(p13.1;q22); CBFB-MYH11; and t(15;17) PML-RARA; and does not include one or more chromosomal abnormalities selected from the group consisting of t(9;11)(p21.3;q23.3); MLLT3-KMT2A; t(6;9)(p23;q34.1); DEK-NUP214; t(v;11q23.3); KMT2A rearranged; t(9;22)(q34.1;q11.2); BCR-ABL1; inv(3)(q21.3q26.2) or t(3;3)(q21.3;q26.2); GATA2,MECOM(EVI1); del(5q); abn(17p); complex karyotype; and monosomal karyotype.

According to some embodiments, the identifying the prognostic group of the subject as intermediate includes the biological sample comprising one or more chromosomal abnormalities selected from the group consisting of t(9;11)(p21;q23); MLLT3-KMT2A; and does not include t(8;21)(q22;q22); RUNX1-RUNX1T1; inv(16)(p13q22) or t(16;16)(p13;q22); CBFB-MYH11; t(15;17) PML-RARA; t(6;9)(p23;q34); DEK-NUP214; t(v;11q23); KMT2A rearranged; t(9;22)(q34;q11); BCR-ABL1; inv(3)(q21q26) or t(3;3)(q21;q26); GATA2,MECOM(EVI1); del(5q); abn(17p); Complex karyotype; and monosomal karyotype.

According to some embodiments, the identifying the prognostic group of the subject as intermediate includes the biological sample comprising one or more chromosomal abnormalities selected from the group consisting of t(9;11)(p21.3;q23.3); MLLT3-KMT2A; and does not include t(8;21)(q22;q22.1); RUNX1-RUNX1T1; inv(16)(p13.1q22) or t(16;16)(p13.1;q22); CBFB-MYH11; t(15;17) PML-RARA; t(6;9)(p23;q34.1); DEK-NUP214; t(v;11q23.3); KMT2A rearranged; t(9;22)(q34.1;q11.2); BCR-ABL1; inv(3)(q21.3q26.2) or t(3;3)(q21.3;q26.2); GATA2,MECOM(EVI1); del(5q); abn(17p); Complex karyotype; and monosomal karyotype.

According to some embodiments, the method provides for determining a prognosis category for a cancer, such as renal carcinoma.

According to some embodiments, the method provides for determining a prognosis category for a cancer, such as colorectal carcinoma.

According to some embodiments, the method provides for determining the prognosis category for a cancer, such as skin cancer.

According to some embodiments, the method provides for determining a prognosis category for a cancer, such as myelodysplastic syndrome (MDS).

According to some embodiments, the method provides for determining the prognosis category for a cancer, such as leukemia.

According to some embodiments, the method provides for determining the prognosis category for a cancer, such as lymphoma.

According to some embodiments, the method provides for determining the prognosis category for a cancer, such as myeloma.

According to some embodiments, the method provides for determining the prognosis category for a cancer, such as tumors of the central nervous system.

According to some embodiments, the method provides for determining the prognosis category for a cancer, such as breast cancer.

According to some embodiments, the method provides for determining the prognosis category for a cancer, such as prostate cancer.

According to some embodiments, the method provides for determining the prognosis category for a cancer, such as cervical cancer.

According to some embodiments, the method provides for determining the prognosis category for a cancer, such as uterine cancer.

According to some embodiments, the method provides for determining the prognosis category for a cancer, such as lung cancer.

According to some embodiments, the method provides for determining the prognosis category for a cancer, such as ovarian cancer.

According to some embodiments, the method provides for determining the prognosis category for a cancer, such as testicular cancer.

According to some embodiments, the method provides for determining the prognosis category for a cancer, such as thyroid cancer.

According to some embodiments, the method provides for determining the prognosis category for a cancer, such as astrocytoma.

According to some embodiments, the method provides for determining the prognosis category for a cancer, such as glioma.

According to some embodiments, the method provides for determining the prognosis category for a cancer, such as pancreatic cancer.

According to some embodiments, the method provides for determining the prognosis category for a cancer, such as mesotheliomas.

According to some embodiments, the method provides for determining the prognosis category for a cancer, such as gastric cancer.

According to some embodiments, the method provides for determining the prognosis category for a cancer, such as liver cancer.

According to some embodiments, the method provides for determining the prognosis category for a cancer, such as renal cancer including nephroblastoma.

According to some embodiments, the method provides for determining the prognosis category for a cancer, such as bladder cancer.

According to some embodiments, the method provides for determining the prognosis category for a cancer, such as oesophageal cancer.

According to some embodiments, the method provides for determining the prognosis category for a cancer, such as cancer of the larynx.

According to some embodiments, the method provides for determining the prognosis category for a cancer, such as cancer of the parotid.

According to some embodiments, the method provides for determining the prognosis category for a cancer, such as cancer of the biliary tract.

According to some embodiments, the method provides for determining the prognosis category for a cancer, such as endometrial cancer.

According to some embodiments, the method provides for determining the prognosis category for a cancer, such as adenocarcinomas.

According to some embodiments, the method provides for determining the prognosis category for a cancer, such as small cell lung carcinoma.

According to some embodiments, the method provides for determining the prognosis category for a cancer, such as neuroblastoma.

According to some embodiments, the method provides for determining the prognosis category for a cancer, such as adrenocortical carcinoma.

According to some embodiments, the method provides for determining the prognosis category for a cancer, such as epithelial carcinoma.

According to some embodiments, the method provides for determining the prognosis category for a cancer, such as desmoid tumors.

According to some embodiments, the method provides for determining the prognosis category for a cancer, such as desmoplastic small round cell tumors.

According to some embodiments, the method provides for determining the prognosis category for a cancer, such as endocrine tumors.

According to some embodiments, the method provides for determining the prognosis category for a cancer, such as Ewing sarcoma family tumors.

According to some embodiments, the method provides for determining the prognosis category for a cancer, such as germ cell tumors.

According to some embodiments, the method provides for determining the prognosis category for a cancer, such as hepatoblastomas.

According to some embodiments, the method provides for determining the prognosis category for a cancer, such as hepatocellular carcinoma.

According to some embodiments, the method provides for determining the prognosis category for a cancer, such as non-rhabdomyosarcome soft tissue sarcomas.

According to some embodiments, the method provides for determining the prognosis category for a cancer, such as osteosarcoma.

According to some embodiments, the method provides for determining the prognosis category for a cancer, such as peripheral primitive neuroectodermal tumors.

According to some embodiments, the method provides for determining the prognosis category for a cancer, such as retinoblastoma.

According to some embodiments, the method provides for determining the prognosis category for a cancer, such as rhabdomyosarcoma.

According to some embodiments, the chromosomal abnormalities is one or more of a deletion, a duplication, or a combination thereof.

According to some embodiments, the reference sample is a sample from one or more subjects having cancer, a sample from one or more subjects not having cancer, or both.

According to one aspect, the present disclosure provides a method for treating a subject with a cancer, the method comprising: a) providing a biological sample from the subject; b) determining copy number of one or more target genes or fragments of the target genes compared to a reference sample by: 1) preparing a cell-free DNA (cfDNA) sample from the biological sample of step a); 2) preparing a sequencing library from the cfDNA from the biological sample, wherein preparing the library comprises consecutive steps of fragmenting the cfDNA, end-repairing, dA-tailing and adaptor ligating the cfDNA fragments; 3) sequencing the adaptor-ligated cfDNA fragments of the one or more target genes or fragments thereof from the sequences of the adaptor-ligated cfDNA fragments for the biological sample and determining copy number of the one or more target genes or fragments thereof 4) comparing, by a computing system, the copy number of the one or more target genes or cfDNA fragments thereof for the biological sample with that of a reference sample, wherein the comparison can determine one or more chromosomal abnormalities in the biological sample; c) identifying the prognostic group of the subject based on step b), wherein the presence of one or more chromosomal abnormalities in the one or more target genes or fragments thereof for the biological sample when compared to the reference sample indicates a subject has a cancer with an adverse, intermediate, or favorable prognosis; d) qualifying the subject for adjunct therapy where the results in (c) indicate an favorable, intermediate or adverse prognosis, and e) administering a therapeutic amount of a chemotherapeutic or immunotherapeutic agent to the subject qualified for adjunct therapy in the favorable, intermediate or adverse prognosis group.

According to some embodiments, the biological sample is a tissue biopsy of the cancer or a liquid biopsy. According to some embodiments, the biological sample is blood, plasma, serum, urine, stool, saliva, tissue, or bodily fluid.

According to some embodiments, the biological sample is plasma derived from peripheral blood that comprises a mixture of cfDNA derived from normal and cancerous cells.

According to some embodiments, the one or more target genes or fragments thereof are selected from Table 3. According to some embodiments, the target gene or fragments thereof are a protein-coding regions of a gene.

According to some embodiments, the sequencing the adaptor-ligated cfDNA fragments of the one or more target genes or fragments thereof from the biological sample includes employing a next generation sequencing (NGS) method.

According to some embodiments, the method provides for determining a prognosis category for a cancer, such as renal carcinoma.

According to some embodiments, the method provides for determining a prognosis category for a cancer, such as colorectal carcinoma.

According to some embodiments, the method provides for determining the prognosis category for a cancer, such as skin cancer.

According to some embodiments, the method provides for determining a prognosis category for a cancer, such as myelodysplastic syndrome (MDS).

According to some embodiments, the method provides for determining the prognosis category for a cancer, such as leukemia.

According to some embodiments, the method provides for determining the prognosis category for a cancer, such as lymphoma.

According to some embodiments, the method provides for determining the prognosis category for a cancer, such as myeloma.

According to some embodiments, the method provides for determining the prognosis category for a cancer, such as tumors of the central nervous system.

According to some embodiments, the method provides for determining the prognosis category for a cancer, such as breast cancer.

According to some embodiments, the method provides for determining the prognosis category for a cancer, such as prostate cancer.

According to some embodiments, the method provides for determining the prognosis category for a cancer, such as cervical cancer.

According to some embodiments, the method provides for determining the prognosis category for a cancer, such as uterine cancer.

According to some embodiments, the method provides for determining the prognosis category for a cancer, such as lung cancer.

According to some embodiments, the method provides for determining the prognosis category for a cancer, such as ovarian cancer.

According to some embodiments, the method provides for determining the prognosis category for a cancer, such as testicular cancer.

According to some embodiments, the method provides for determining the prognosis category for a cancer, such as thyroid cancer.

According to some embodiments, the method provides for determining the prognosis category for a cancer, such as astrocytoma.

According to some embodiments, the method provides for determining the prognosis category for a cancer, such as glioma.

According to some embodiments, the method provides for determining the prognosis category for a cancer, such as pancreatic cancer.

According to some embodiments, the method provides for determining the prognosis category for a cancer, such as mesotheliomas.

According to some embodiments, the method provides for determining the prognosis category for a cancer, such as gastric cancer.

According to some embodiments, the method provides for determining the prognosis category for a cancer, such as liver cancer.

According to some embodiments, the method provides for determining the prognosis category for a cancer, such as renal cancer including nephroblastoma.

According to some embodiments, the method provides for determining the prognosis category for a cancer, such as bladder cancer.

According to some embodiments, the method provides for determining the prognosis category for a cancer, such as oesophageal cancer.

According to some embodiments, the method provides for determining the prognosis category for a cancer, such as cancer of the larynx.

According to some embodiments, the method provides for determining the prognosis category for a cancer, such as cancer of the parotid.

According to some embodiments, the method provides for determining the prognosis category for a cancer, such as cancer of the biliary tract.

According to some embodiments, the method provides for determining the prognosis category for a cancer, such as endometrial cancer.

According to some embodiments, the method provides for determining the prognosis category for a cancer, such as adenocarcinomas.

According to some embodiments, the method provides for determining the prognosis category for a cancer, such as small cell lung carcinoma.

According to some embodiments, the method provides for determining the prognosis category for a cancer, such as neuroblastoma.

According to some embodiments, the method provides for determining the prognosis category for a cancer, such as adrenocortical carcinoma.

According to some embodiments, the method provides for determining the prognosis category for a cancer, such as epithelial carcinoma.

According to some embodiments, the method provides for determining the prognosis category for a cancer, such as desmoid tumors.

According to some embodiments, the method provides for determining the prognosis category for a cancer, such as desmoplastic small round cell tumors.

According to some embodiments, the method provides for determining the prognosis category for a cancer, such as endocrine tumors.

According to some embodiments, the method provides for determining the prognosis category for a cancer, such as Ewing sarcoma family tumors.

According to some embodiments, the method provides for determining the prognosis category for a cancer, such as germ cell tumors.

According to some embodiments, the method provides for determining the prognosis category for a cancer, such as hepatoblastomas.

According to some embodiments, the method provides for determining the prognosis category for a cancer, such as hepatocellular carcinoma.

According to some embodiments, the method provides for determining the prognosis category for a cancer, such as non-rhabdomyosarcome soft tissue sarcomas.

According to some embodiments, the method provides for determining the prognosis category for a cancer, such as osteosarcoma.

According to some embodiments, the method provides for determining the prognosis category for a cancer, such as peripheral primitive neuroectodermal tumors.

According to some embodiments, the method provides for determining the prognosis category for a cancer, such as retinoblastoma.

According to some embodiments, the method provides for determining the prognosis category for a cancer, such as rhabdomyosarcoma.

According to some embodiments, the chromosomal abnormalities is one or more of a deletion, a duplication, or a combination thereof.

According to some embodiments, the reference sample is a sample from one or more subjects having cancer, a sample from one or more subjects not having cancer, or both.

According to some embodiments, the step b-3) comprises determining sequence read depth of one or more target segments and one or more non-target segments.

According to some embodiments, the identifying the prognostic group of the subject as adverse includes the biological sample comprising one or more chromosomal abnormalities selected from the group consisting of t(6;9)(p23;q34); DEK-NUP214; t(v;11q23); KMT2A rearranged; t(9;22)(q34;q11); BCR-ABL1; inv(3)(q21q26) or t(3;3)(q21;q26); GATA2,MECOM(EVI1); del(5q); abn(17p); Complex karyotype; and monosomal karyotype.

According to some embodiments, the identifying the prognostic group of the subject as adverse includes the biological sample comprising one or more chromosomal abnormalities selected from the group consisting of t(6;9)(p23;q34.1); DEK-NUP214; t(v;11q23.3); KMT2A rearranged; t(9;22)(q34.1;q11.2); BCR-ABL1; inv(3)(q21.3q26.2) or t(3;3)(q21.3;q26.2); GATA2,MECOM(EVI1); del(5q); abn(17p); Complex karyotype; and monosomal karyotype.

According to some embodiments, the identifying the prognostic group of the subject as favorable includes the biological sample comprising one or more chromosomal abnormalities selected from the group consisting of t(8;21)(q22;q22); RUNX1-RUNX1T1; inv(16)(p13q22) or t(16;16)(p13;q22); CBFB-MYH11; and t(15;17) PML-RARA; and does not include one or more of chromosomal abnormalities selected from the group consisting of t(9;11)(p21;q23); MLLT3-KMT2A; t(6;9)(p23;q34); DEK-NUP214; t(v;11q23); KMT2A rearranged; t(9;22)(q34;q11); BCR-ABL1; inv(3)(q21q26) or t(3;3)(q21;q26); GATA2,MECOM(EVI1); del(5q); abn(17p); Complex karyotype; and monosomal karyotype.

According to some embodiments, the identifying the prognostic group of the subject as favorable includes the biological sample comprising one or more chromosomal abnormalities selected from the group consisting of t(8;21)(q22;q22.1); RUNX1-RUNX1T1; inv(16)(p13.1q22) or t(16;16)(p13.1;q22); CBFB-MYH11; and t(15;17) PML-RARA; and does not include one or more of chromosomal abnormalities selected from the group consisting of t(9;11)(p21.3;q23.3); MLLT3-KMT2A; t(6;9)(p23;q34.1); DEK-NUP214; t(v;11q23.3); KMT2A rearranged; t(9;22)(q34.1;q11.2); BCR-ABL1; inv(3)(q21.3q26.2) or t(3;3)(q21.3;q26.2); GATA2,MECOM(EVI1); del(5q); abn(17p); Complex karyotype; and monosomal karyotype.

According to some embodiments, the identifying the prognostic group of the subject as intermediate includes the biological sample comprising one or more chromosomal abnormalities selected from the group consisting of t(9;11)(p21;q23.3); MLLT3-KMT2A; and does not include t(8;21)(q22;q22); RUNX1-RUNX1T1; inv(16)(p13q22) or t(16;16)(p13;q22); CBFB-MYH11; t(15;17) PML-RARA; t(6;9)(p23;q34); DEK-NUP214; t(v;11q23); KMT2A rearranged; t(9;22)(q34;q11); BCR-ABL1; inv(3)(q21q26) or t(3;3)(q21;q26); GATA2,MECOM(EVI1); del(5q); abn(17p); complex karyotype; and monosomal karyotype.

According to some embodiments, the identifying the prognostic group of the subject as intermediate includes the biological sample comprising one or more chromosomal abnormalities selected from the group consisting of t(9;11)(p21.3;q23.3); MLLT3-KMT2A; and does not include t(8;21)(q22;q22.1); RUNX1-RUNX1T1; inv(16)(p13.1q22) or t(16;16)(p13.1;q22); CBFB-MYH11; t(15;17) PML-RARA; t(6;9)(p23;q34.1); DEK-NUP214; t(v;11q23.3); KMT2A rearranged; t(9;22)(q34.1;q11.2); BCR-ABL1; inv(3)(q21.3q26.2) or t(3;3)(q21.3;q26.2); GATA2,MECOM(EVI1); del(5q); abn(17p); complex karyotype; and monosomal karyotype.

### EXAMPLES

### Example 1

### Liquid Biopsy Positive Rate for Detecting Chromosomal Structural Abnormalities

### Patient Samples

Using 2821 peripheral blood samples collected over approximately 18 month collected in EDTA anticoagulant we extracted cfDNA using The Maxwell® RSC 48 instruments. DNA was extracted from separated plasma within 48 hours of collection. Study data was collected under approved IRB (WCG IRB # 1-1476184-1).

### Next Generation Sequencing

The extracted DNA was sequenced using 100 ng of DNA. The Library for targeted 275 gene sequencing is based on Single Primer Extension (SPE) chemistry. The DNA sequencing includes all coding exons of the 275 genes. For each exon approximately 50 intronic nucleotides were also sequenced. Genomic DNA samples were end repaired and A-tailed, then unique medical identifiers (UMIs) and sample index were added. Target enrichment was performed post-UMI assignment to ensure that DNA molecules containing UMIs were sufficiently enriched in the sequenced library. For enrichment, ligated DNA molecules were subjected to several cycles of targeted polymerase chain reaction (PCR) using one region-specific primer and one universal primer complementary to the adapter. A universal PCR was ultimately carried out to amplify the library and add platform-specific adapter sequences and additional sample indices. The sequencing was conducted using the Illumina NextSeq 550 instruments. The targeted 275 genes are listed in Table 3.

TABLE 3
Targeted Genes

ABL1
ACVR1B
AKT1
AKT2
AKT3
ALK
AMER1
APC
AR
ARAF
ARID1A
ARID1B
ARID2
ASXL1
ATM
ATR
ATRX
AURKA
AURKB
AURKC
AXIN1
AXIN2
B2M
BAP1
BCL2
BCL2L1
BCL6
BCOR
BCORL1
BCR
BIRC3
BLM
BRAF
BRCA1
BRCA2
BRIP1
BTK
CALR
CARD11
CBL
CBLB
CBLC
CCND1
CCND3
CCNE1
CD274
CD79A
CD79B
CDC73
CDH1
CDK12
CDK4
CDK6
CDKN2A
CDKN2B
CDKN2C
CEBPA
CHEK1
CHEK2
CIC
CREBBP
CRLF2
CSF1R
CSF3R
CTCF
CTNNA1
CTNNB1
CUX1
CXCR4
CYLD
DAXX
DDR2
DICER1
DNM2
DNMT3A
DOT1L
EED
EGFR
EGLN1
EP300
EPAS1
EPHA3
EPHA5
ERBB2
ERBB3
ERBB4
ERG
ESR1
ETV6
EXO1
EZH2
FAM175A
FAM46C
FANCA
FANCC
FANCD2
FANCE
FANCF
FANCG
FAS
FBXW7
FGF4
FGF6
FGFR1
FGFR2
FGFR3
FGFR4
FH
FLCN
FLT3
FLT4
FOXL2
FUBP1
GALNT12
GATA1
GATA2
GATA3
GEN1
GNA11
GNAQ
GNAS
GREM1
GRIN2A
H3F3A
HGF
HIST1H3B
HNF1A
HOXB13
HRAS
HSP90AA1
ID3
IDH1
IDH2
IGF1R
IKZF1
IKZF3
IL7R
INHBA
IRF4
JAK1
JAK2
JAK3
KAT6A
KDM5C
KDM6A
KDR
KEAP1
KIT
KMT2A
KMT2B
KMT2C
KMT2D
KRAS
LRP1B
MAP2K1
MAP2K2
MAP2K4
MAP3K1
MAP3K14
MAPK1
MCL1
MDM2
MDM4
MED12
MEF2B
MEN1
MET
MITF
MLH1
MPL
MRE11A
MSH2
MSH6
MTOR
MUTYH
MYC
MYCL
MYCN
MYD88
NF1
NF2
NFE2L2
NFKBIA
NKX2-1
NOTCH1
NOTCH2
NOTCH3
NPM1
NRAS
NSD1
NTRK1
NTRK2
NTRK3
PAK3
PALB2
PAX5
PBRM1
PDGFRA
PDGFRB
PHF6
PIK3CA
PIK3R1
PIK3R2
PIM1
PLCG1
PMS1
PMS2
POLD1
POLE
PPM1D
PPP2R1A
PRDM1
PRKAR1A
PRKDC
PRSS1
PTCH1
PTEN
PTPN11
RAC1
RAD21
RAD50
RAD51
RAF1
RB1
RET
RHEB
RHOA
RIT1
RNF43
ROS1
RUNX1
SDHB
SETBP1
SETD2
SF3B1
SMAD2
SMAD4
SMARCA4
SMARCB1
SMC1A
SMC3
SMO
SOCS1
SOX2
SOX9
SPOP
SRC
SRSF2
STAG2
STAT3
STK11
SUFU
SUZ12
TAL1
TCF3
TERT
TET2
TGFBR2
TNFAIP3
TNFRSF14
TP53
TRAF3
TSC1
TSC2
TSHR
U2AF1
U2AF2
VHL
WHSC1
WT1
XPO1
XRCC2
XRCC3
ZNF217
ZRSR2

### Using CNVKit for Copy Number Detection

The CNVkit software was implemented to evaluate CNV in the analyzed samples. Briefly, the software takes advantage of both on- and off-target sequencing reads, compares binned read depths in on- and off-target regions to pooled normal reference, and estimates the copy number at various resolutions.

### Patients and Diseases

Between March 2020 and September 2021, we tested 2821 cfDNA from peripheral blood samples from patients with hematologic neoplasms or expected to have hematologic neoplasms for the presence of circulating tumor DNA (ctDNA). This included patients with various types of lymphoma and myeloid neoplasms. Some of the samples were testing at initial diagnosis and some for follow up and minimal residual disease detection.

### Results

Of the tested 2821 samples, 1539 (54.5%) showed evidence of mutation consistent with the presence of neoplastic clone. Of the 1539 positive samples, 633 (41%) had abnormalities associated with lymphoid neoplasms and 906 (59%) had myeloid neoplasms. The median variant allele frequency (VAF) was 8.09% and the range was 0.002% to 99.55%. Of the lymphoid neoplastic cases, 76 (12%) had chromosomal abnormalities detected in cfDNA. Of the 906 myeloid cases, 146 (16%) samples showed chromosomal structural abnormalities. Results are shown in Table 4.

TABLE 4

| | Lymphoid | Myeloid | Total |
|---|---|---|---|
| Mutation positive | 633 (41%) | 906 (59%) | 1539 |
| Chromosomal abnormalities | 76 (12%) | 146 (16%) | 222 (14% |
| No mutations | 1 | 1 | 2 |

Two samples showed no detectable mutations, but showed chromosomal structural abnormalities. One myeloid case showed a 5q deletion only without mutations. This case was diagnosed as an isolated 5q deletion syndrome based cfDNA analysis and was confirmed by bone marrow morphology and cytogenetic studies. The second case was chronic lymphocytic leukemia that showed no evidence of mutation, but trisomy 12 was detected on cfDNA analysis.

### Example 2

### Sensitivity of NGS in Detecting Chromosomal Structural Abnormalities

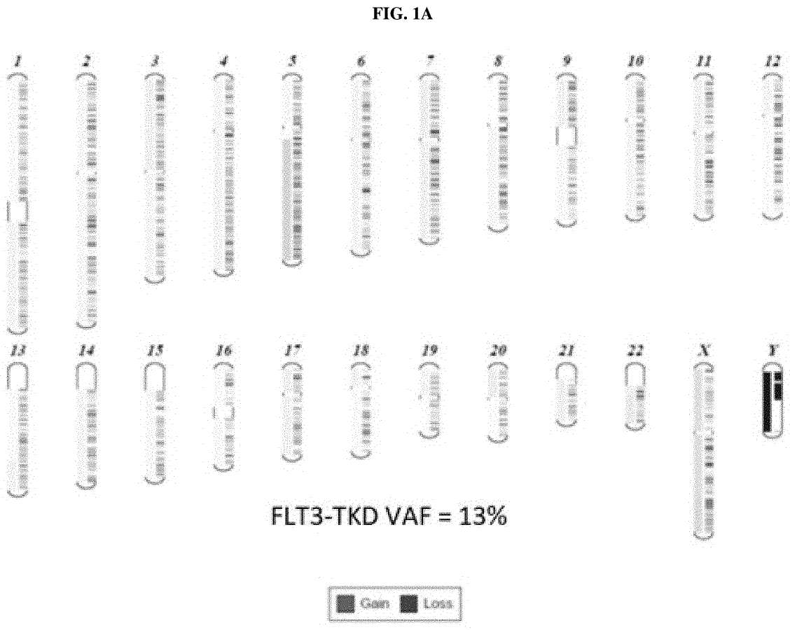

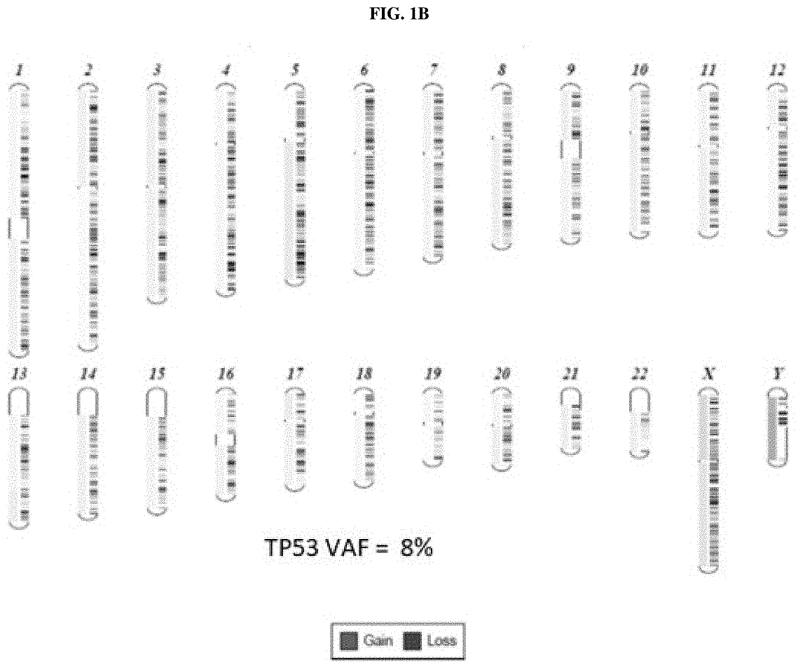

In order to evaluate the sensitivity of liquid biopsy in detected chromosomal abnormalities, we evaluated the variant allele frequency (VAF) in the cases with demonstrable chromosomal abnormalities in the cfDNA. The VAF varied significantly between 0.002% and 99.55% dependent on the type of mutation and the expected heterogeneity within the same sample. However, samples with VAF detected at 13% or higher were associated with clear chromosomal detectable chromosomal structural abnormalities (see FIG. 1A). Lower levels to a limit of 8% showed some changes that can be interpreted as possibility of the chromosomal abnormality (FIG. 1B). Lower VAF in a sample can be considered below the level necessary for detecting specific CNV. Low VAF might increase if higher number of genes are used to cover larger segments of the chromosomes. Mathematical reliability of detecting chromosomal abnormalities increases significantly with increasing coverage of chromosomes. The exception for this is when the neoplasm is driven by chromosomal abnormalities such as in isolated 5q deletion syndrome and some chronic lymphocytic leukemia and rare cases of other neoplasms or neoplasms with fusion genes.

### Example 3

### Correlation between NGS Chromosomal Structural Analysis and Bone Marrow Cytogenetic Studies

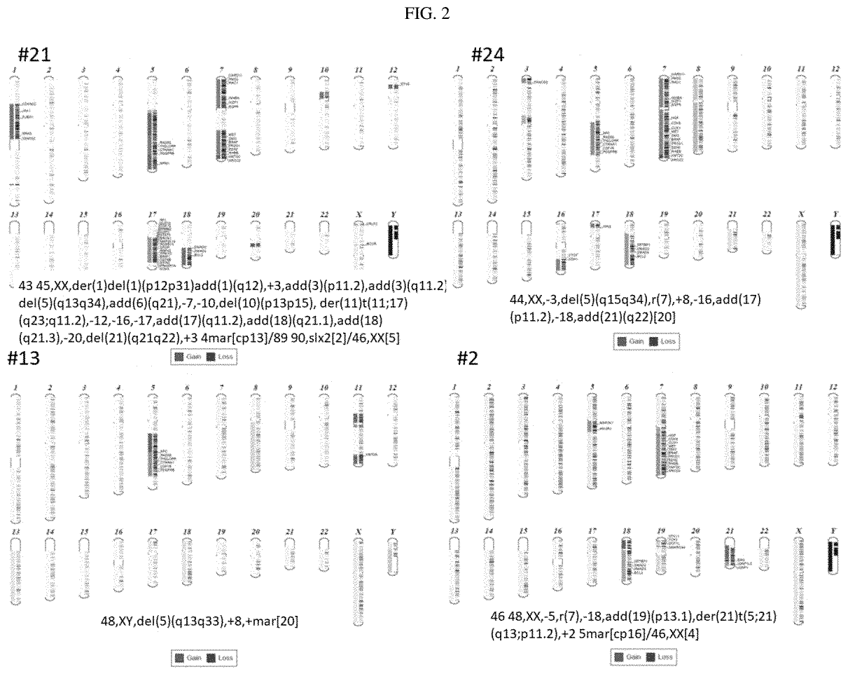

In order to confirm the clinical reliability of detected chromosomal structural abnormalities using liquid biopsy and targeted gene panel, we compared findings with conventional cytogenetics performed on bone marrow samples. Eighty nine liquid biopsy samples had bone marrow cytogenetic data obtained within a week or two of the liquid biopsy sample. As shown in Table 5, there were 33 samples (37%), 3 samples (3%) with fusion only abnormality, 8 samples (9%) with “no metaphases detected”, and 45 (51%) with normal karyotype. Since the NGS design is to detect chromosomal gain or loss, as expected the 4 cases with fusion genes were not called by NGS. Three of the “no metaphases detected” cases had cytogenetic abnormalities detected by NGS (Table 5) and 5 samples showed no chromosomal loss or gain by NGS. As expected, there was a difference in the description of the abnormalities as detected by cytogenetics when compared with those detected by NGS. FIG. 2 illustrate some examples of differences between findings reported by cytogenetics and the findings by NGS testing (FIG. 2). Simple abnormalities such as trisomies and monosomies were called in similar fashion, but some complex findings such as isochromosomes, derivative and dicentric chromosomes were called differently. One case, which showed only tetraploid metaphases, appeared as normal on NGS sequencing. The NGS resolved the marker chromosomes that are described in the cytogenetic analysis.

To compare the two approaches, the cytogenetic and chromosomal findings of the 89 cases were grouped into three myeloid risk groups: intermediate, poor and complex. This classification showed 100% concordance between the NGS chromosomal structural analysis and cytogenetic data. Results are shown in Table 5.

TABLE 5

| | CNV by | | Interpretation |
|---|---|---|---|
| Sample | Liquid | | (agreement: |
| # | Biopsy NGS | Cytogenetic Report | Yes/No) |
| 1 | 5q−, 7q−, | 46 48, XX, −5, r(7), −18, | complex |
| | 18p−, 19p+, | add(19)(p13.1), der(21)t(5; 21)(q13; | (Yes) |
| | 19q+, 21q+ | p11.2), +2 5mar[cp16]/46, XX[4] | |
| | (amplification) | | |
| 2 | 5q−, 7q−, −18 | 46 48, XX, −5, r(7), −18, add(19)(p13.1), | complex |
| | (bi-allelic | der(21)t(5; 21)(q13; p11.2), +2 | (Yes) |
| | 18p−), 19p+, | 5mar[cp16]/46, XX[4] | |
| | 19q+, 21q+ | | |
| | (amplification) | | |
| 3 | 1q+, 7q−, | 46, XY, +1, der(1; 7)(q10; p10)[2]/47, idem, +21 | complex |
| | 19q+, 21q+ | [14]/48, idem, +8, +21[2]/47, | (Yes) |
| | | idem, +8[1]/46, XY[1] | |
| 4 | 5q−, 8q+, | 46, XX, del(3)(q21q25), add(5)(p13), | poor |
| | and 17p− | del(5)(q13q33), −17, der(17; 21)(q10; | (Yes) |
| | | q10), add(21)(p11.2), +mar[cp20] | |
| 5 | 5q−, 7q−, +8, | 46 47, XY, del(5)(q15q34), add(7)(q21), +8, | poor |
| | 17p−, 17q1 | del(11)(q22q23), −17, −19, −22, +2 | (Yes) |
| | (proximal), 17q+ | 3mar[15]/46, XY[5] | |
| | (distal), −19, +21 | | |
| 6 | 5q−; 7q−, | 45, XX, del(5)(q13q33), −7, | complex |
| | 17p−, 18q− | der(17)add(17)(p11.1)add(17)(q23), | (Yes) |
| | | del(18)(q21.3q23)[12]/45, XX, del(5)(q13q33), | |
| | | dic(7; 17)(q11.2; p11.1), | |
| | | del(18)(q21.3q23)[3]/46, XX[5] | |
| 7 | 2p−, 3p−, 5q−, | 42 43, XY, add(3)(p13), −2, −3, −5, | complex |
| | 17p−, 17q− | add(16)(q12.1), −17, add(19)(q13.1), −20, +2mar, | (Yes) |
| | (partial), | inc[cp3] LIMITED STUDY | |
| | 19p+, 21q+ | | |
| 8 | 3p−, 5q−, 7q−, | 45, X, −Y, add(1)(q21), del(3)(p13p25), dic(5; | complex |
| | 12p− and +22. | 12)(q11.12; p11.2), add(7)(q11.2), +mar[19]/46, XY[1] | (Yes) |
| 9 | monosomy 7 | 45, XX, −7[9]/46.xx[11] | poor |
| | | | (Yes) |
| 10 | 5q−, +8, +11, | 40 48, XY, +5, del(5)(q15q33), der(5; 14)(p10; q10), | complex |
| | +13, and 17q− | der(5; 17)(p10; q10), −7, +8, i(11)(q10), | (Yes) |
| | | idic(13)(p11.2), add(14)(p11.2), add(16)(q24), | |
| | | i(17)(q10), −18, der(20)t(11; 20)(q13; | |
| | | q13.3), −21, +22, +mar[cp17]/46, XY[3] | |
| 11 | 5q−, −7, | 44, XY, −5, −7, inv(12)(p13q13), add(17)(p11.2), −18, | complex |
| | 11q+, 12p−, | der(19)ins(19; ?) (q13.1; ?), −20, +2mar[8]/43, idem, −11, | (Yes) |
| | 17p−, 18p−, | add(13)(q32)[5]/44, idem, r(11)(p15q25)[2]/46, XY[7] | |
| | 19q+ | | |
| 12 | −5, 8p+, 9p−, | 45, XX, add(3)(q21), add(5) (q11.2)x2, | complex |
| | 11p−, 17p−, −18 | der(6)t(6; 17) (q27; q11.2), add(7) (q31), +8, | (Yes) |
| | and 20p− | der(13)t(5; 13) (q15; q32), −17, 1-18[10]/46, | |
| | | XX[4] INCOMPLETE STUDY | |
| 13 | 5q−, 8q+, | 48, XY, del(5)(q13q33), +8, +mar[20] | poor |
| | 11p+ (proximal | | (Yes) |
| | amplification), | | |
| | 11q (KMT2A gene | | |
| | amplification), +13. | | |
| 14 | 3p−, 5q−, −7, | 43 44, XX, −3, dic(5; 15)(q11.2; p11.2), −6, | complex |
| | and 12p− | del(6)(p23p25), der(7; 12)t(7; 12)(p10; | (Yes) |
| | | q10)ins(7; ?)(p11.2; ?), +der(?)t(?; 3)(?; | |
| | | q12), +r[cp17]/46, XX[3] | |
| 15 | 4q−, 5q−, | 44 50, XYY?c, −4, dic(5; 17)(q13; p11.2), add(7)(q11.2), | complex |
| | 7q−, +11, | der(12) ins(12; ?)(q13; ?), −14, −21, −22, +2 | (Yes) |
| | 12p−, 13p+, | 8mar[cp14]/47 48, idem, +11, +13[cp3]/81 | |
| | 13q+, −16, | 89, idemx2[2]/47, XYY?c[8] | |
| | 17p−, −18, | | |
| | +21 and +Y. | | |
| 16 | 7q− | 46, XX, del(7)(q22q32)[19]/46, XX[1] | poor |
| | | | (Yes) |
| 17 | 12p− | 46, XY[20] | intermediate |
| | | | (Yes) |
| 18 | 1q+, 2p+, | 44 46, XY, add(2)(p11.2), −3, | complex |
| | 3q−, −4, −7, | add(3)(q11.2), add(4)(q12), −5, | (Yes) |
| | 9q+, 13q+, | der(5)t(3; 5)(p13; p13)ins(5; ?)(p13; ?), | |
| | 17p−, 17q−, | del(6)(p23p25), −9, add(13)(q12), −17, −17, | |
| | 20q+, 21q− | add(20)(q11.2), +3 5mar, inc[cp5] | |
| | | Limited Study | |
| 19 | 1q+ and | 46, XY, i(14)(q10)[20] | intermediate |
| | trisomy 14. | | (Yes) |
| 20 | 8p−, 9p− | 45, XY, der(8)t(1; 8)(q12; p21), inv(8)(p11, 2q24.3), | poor |
| | (PAX5, | add(9)(p13), i(17)(q10), −20[4]/46, XY[20] | (Yes) |
| | CD174, | | |
| | CDKN2A/B), | | |
| | 17p− and | | |
| | gain: 1p+, | | |
| | 17p+. | | |
| 21 | 1p−, 5q−, −7, | 43 45, XX, der(1)del(1)(p12p31)add(1)(q12), +3, | complex |
| | 10p−, 12p−, | add(3)(p11.2), add(3)(q11.2), del(5)(q13q34), | (Yes) |
| | 17q−, 18q− | add(6)(q21), −7, −10, del(10)(p13p15), | |
| | and 20q− | der(11)t(11; 17)(q23; q11.2), −12, −16, −17, | |
| | | add(17)(q11.2), add(18)(q21.1), add(18)(q21.3), −20, | |
| | | del(21)(q21q22), +3 4mar[cp13]/89 90, slx2[2]/46, XX[5] | |
| 22 | 3q−, 5q−, 7p−, | 43, XY, del(5)(q13q33), del(7)(p13p22), add(9) (q13), | complex |
| | 8q+, 12p−, | der(12)add(12)(p11.2)del(12)(q14q21), −16, −18, −20[20] | (Yes) |
| | 12q−, −16, | | |
| | 17p−, −18, −20 | | |
| 23 | +8 and 10q− | 46, XY, der(4)t(4; 8)(q33; q13), t(8; 21)(q22; q22)[7]/47, | intermediate |
| | | XY, +8, t(8; 21)(q22; q22)[5], LIMITED ANALYIS | (Yes) |
| 24 | 3p−, 5q−, −7, +8, | 44, XX, −3, del(5)(q15q34), r(7), +8, −16, | complex |
| | 16q−, 17p−, | add(17)(p11.2), −18, add(21)(q22)[20] | (Yes) |
| | 18q−, +21 | | |
| 25 | Trisomy 21 | 47, XX, +21[13]/46, XX[3] | Intermediate |
| | | | (Yes) |
| 26 | 8p+, 18p− | 47, XY, +8[4]/46, XY[16] | Intermediate |
| | | | (Yes) |
| 27 | 1q+, 8q+ | 47, XY, dup(1)(q11q44), +8[17]/46, XY[3] | Intermediate |
| | | | (Yes) |
| 28 | 1p+, 5q−, +6, | 44, XY, add(2)(p11.2), der(5)t(5; 17)(q15; q21), add(6)(p21.3), | Complex |
| | 7q−, −11, 17p− | del(7) (q22q36), −11, del(13)(q12q14), −14, der(16)t(14; | (Yes) |
| | and others | 16)(q24; q11.2), −17, add(21)(q22), +r[19]/46, XY[1] | |
| 29 | monosomy 7 | 46, XY, r(7)[3]/46, XY[1], | Poor |
| | and 12p− | LIMITED ANALYSIS | (yes |
| 30 | Normal | 92, XXXX, add(12)(q24.1)x2[20] | Intermediate |
| | | | (Yes) |
| 31 | 9p− | 46, XY, del(9)(q13q34)[3]/46, XY[17] | Intermediate |
| | | | (Yes) |
| 32 | 2q+, 2q− | 39 45, XX, add(2)(q22), del(3)(p11), dic(5; 7)(q11.2; | Complex |
| | (distal, IDH1 | q11.1), −12, −17, +mar[cp19]/46, XX[1] | (Yes) |
| | and ERBB4 | | |
| | deletion), 3p−, | | |
| | 5q−, 7q−, | | |
| | and 12p−. | | |
| 33 | 5q+ (gain) | 46, | intermediate |
| | | XX[20] | (Yes) |
| 34 | 9p− (deletion | NO METAPHASES DETECTED | Poor |
| | of CDKN2A | | (N/A) |
| | and CDKN2B) | | |
| 35 | 7q− and 8q+ | NO METAPHASES DETECTED | Poor |
| | | | (N/A) |
| 36 | 12p− | NO METAPHASES DETECTED | Intermediate |
| | | | (N/A) |
| 37 | normal | No Metaphases Detected | Intermediate |
| | | | (N/A) |
| 38 | Normal | NO METAPHASES DETECTED | Intermediate |
| | | | (N/A) |
| 39 | Normal | NO METAPHASES DETECTED | Intermediate |
| | | | (N/A) |
| 40 | Normal | No Metaphases Detected | Intermediate |
| | | | (N/A) |
| 41 | Normal | NO METAPHASES DETECTED | Intermediate |
| | | | (N/A) |
| 42 | Normal | 46, XX, t(9; 22)(q34; q11.2)[4]/46, XX[16] | No Loss/Gain |
| | | | (Yes) |
| 43 | Normal | 46, XX, inv(16)(p13.1q22)[22] | No Loss/Gain |
| | | | (Yes) |
| 44 | Normal | 46, XY, t(9; 22)(q34; q11.2)[20] | No Loss/Gain |
| | | | (Yes) |
| | 45 sample with | 45 samples with Normal Karyotype | 45 sample |
| | no chromosomal | | (Yes) |
| | abnormalities | | |

### Example 4

### Network

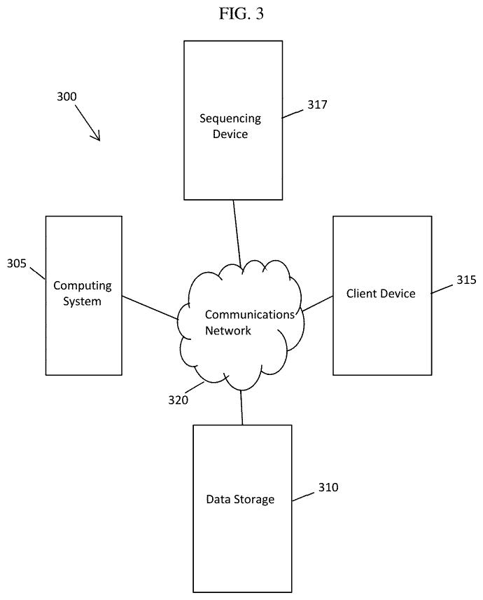

FIG. 3 schematically depicts a network 300 for implementing some aspects in accordance with some embodiments. Network 300 may include a computing system 305, a client device 315, and data storage 310. In some embodiments, network 300 may also include a sequencing device 317. In some embodiments, computing system 305, client device 315, sequencing device 317, and/or data storage 310 may be connected to network 320. However, in other embodiments, two or more of computing system 305, client device 315, sequencing device 317, and/or data storage 310 may be connected directly with each other, without network 320. While one computing system 305, one client device 315, one sequencing device 317, and one data storage 310 are shown in FIG. 3, it should be appreciated that any number of computing systems, client devices, sequencing devices, and data storages could be used.

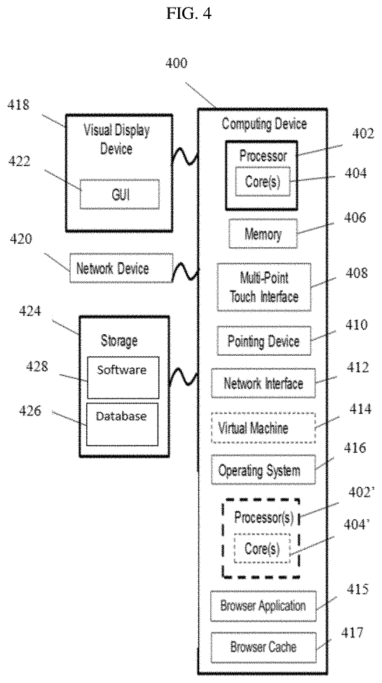

Computing system 305 may include one or more computing devices configured to perform one or more operations consistent with disclosed embodiments. Computing system 305 is further described in connection with FIG. 4. In some embodiments, computing system 305 may perform at least some aspects or steps of the described methods for selecting subjects diagnosed with cancer or suspected of having cancer for different prognostic groups or falling within a treatable prognostic group. In some embodiments, computing system 305, sequencing device 317 and/or client device 315 may perform at least some aspects or steps of the described methods in some embodiments. For example, in some embodiments, sequencing device 317 is employed for sequencing the adaptor-ligated DNA fragments of one or more target genes or fragments thereof in a biological sample and/or in a reference sample. In some embodiments, sequencing device 317 and computing system 305 are employed for sequencing the adaptor-ligated DNA fragments of one or more target genes or fragments thereof in a biological sample and/or in a reference sample.

In some embodiments, computing system 305 is employed for determining a copy number of one or more target genes or fragments of a biological sample and/or of a reference sample from the sequences of the adaptor-ligated DNA fragments for the biological sample and/or for the reference sample. In some embodiments, computing system 305 and/or client device 315 are employed for determining a copy number of one or more target genes or fragments of a sample and/or of a reference sample from the sequences of the adaptor-ligated DNA fragments for the biological sample and/or for the reference sample.

In some embodiments, computing system 305 compares copy number of the one or more target genes or cfDNA fragments thereof for the biological sample with that of a reference sample where the comparison can determine one or more chromosomal abnormalities in the biological sample. In some embodiments, computing system 305 and/or client device 315 compare copy number of the one or more target genes or cfDNA fragments thereof for the biological sample with that of a reference sample where the comparison can determine one or more chromosomal abnormalities in the biological sample.

In some embodiments, computing system 305 identifies the prognostic group of the subject based on the presence of the one or more chromosomal abnormalities in the one or more target genes or fragments thereof for the biological samples when compared to the reference sample. In some embodiments, computing system 305 and/or client device 315 identify the prognostic group of the subject based on the presence of the one or more chromosomal abnormalities in the one or more target genes or fragments thereof for the biological samples when compared to the reference sample.

In some embodiments, computing system 305 identifies the subject as a candidate for chemotherapy or immunotherapy based on the intermediate or complex prognosis. In some embodiments, computing system 305 and/or client device 315 identify the subject as a candidate for chemotherapy or immunotherapy based on the intermediate or complex prognosis.

In some embodiments, computing system 305 and/or client device 315 display (e.g., in a graphical user interface) an identification of or information regarding an identification of the prognostic group of the subject. In some embodiments, computing system 305 and/or client device 315 display (e.g., in a graphical user interface) an identification of the subject as a candidate for chemotherapy or immunotherapy based on the intermediate or complex prognosis. In some embodiments, computing system 305 and/or client device 315 display (e.g., in a graphical user interface) a recommendation of immunotherapy or chemotherapy for the subject based on the intermediate or complex prognosis.

In some embodiments, computing system 305 and/or client device 315, transmit an identification of or information regarding an identification of the prognostic group of the subject. In some embodiments, computing system 305 and/or client device 315 transmit an identification of the subject as a candidate for chemotherapy or immunotherapy based on the intermediate or complex prognosis. In some embodiments, computing system 305 and/or client device 315 transmit a recommendation of immunotherapy or chemotherapy for the subject based on the intermediate or complex prognosis.

In some embodiments, computing system 305, client device 315, data storage 310, or a combination of the aforementioned store an identification of or information regarding an identification of the prognostic group of the subject. In some embodiments, computing system 305, client device 315, data storage 310, or a combination of the aforementioned store an identification of or information regarding an identification of the subject as a candidate for chemotherapy or immunotherapy. In some embodiments, computing system 305, client device 315, data storage 310, or a combination of the aforementioned store a recommendation of immunotherapy or chemotherapy for the subject or information regarding a recommendation of immunotherapy or chemotherapy for the subject.

Data storage 305 may include one or more computing devices configured with appropriate software to perform operations consistent with storing and providing data. Data storage 305 may include, for example, Oracle™ databases, Sybase™ databases, or other relational databases or non-relational databases, such as Hadoop™ sequence files, HBase™ or Cassandra™. Data storage 305 may include computing components (e.g., database management system, database server, etc.) configured to receive and process requests for data stored in memory devices of data storage 305 and to provide data from data storage 305. In some embodiments, data storage 305 may be configured to store the dataset including cell-free DNA sequencing data used by computing system 305. In some embodiments, data storage 305 may be configured to store CNVkit software used by computing system 305. While data storage 305 is shown separately, in some embodiments, data storage 305 may be included in or otherwise related to computing system 305 and/or client device 315.

Client device 315 may include a desktop computer, a laptop, a server, a mobile device (e.g., tablet, smart phone, etc.), a wearable computing device, or other type of computing device. Client device 315 may include one or more processors configured to execute software instructions stored in memory, such as memory included in client device 315. In some embodiments, client device 315 may include software that when executed by a processor performs known Internet-related communication and content display processes. For instance, client device 315 may execute browser software that generates and displays interfaces including content on a display device included in, or connected to, client device 315. Client device 315 may execute applications that allows client device 315 to communicate with components over network 170 and generate and display content in interfaces via display devices included in client device 315. For example, client device 315 may display results produced by computing system 305, such as qualified subjects for chemotherapy or immunotherapy. Computing system 305 may communicate the results to the client device 315.

Computing system 305, client device 315, and database 315 are shown as a different components. However, computing system 305, client device 315, and/or database 315 may be implemented in the same computing system or device. For example, computing system 305, client device 315, and/or database 315 may be embodied in a single computing device.

Network 320 may be any type of network configured to provide communications between components of network 320. For example, network 320 may be any type of network (including infrastructure) that provides communications, exchanges information, and/or facilitates the exchange of information, such as the Internet, a Local Area Network, near field communication (NFC), optical code scanner, or other suitable connection(s) that enables the sending and receiving of information between the components of network 320. In other embodiments, one or more components of network 320 may communicate directly through a dedicated communication link(s).

FIG. 4 schematically depicts a computing system 400 for implementing some aspects in accordance with some embodiments. In some embodiments, computing device 400 may be computing system 305 shown in FIG. 3. In some embodiments, computing device 400 may be client device 315 shown in FIG. 3. Computing device 400 includes one or more non-transitory computer-readable media for storing one or more computer-executable instructions or software for implementing exemplary embodiments. The non-transitory computer-readable media can include, but are not limited to, one or more types of hardware memory, non-transitory tangible media (for example, one or more magnetic storage disks, one or more optical disks, one or more USB flash drives), and the like. For example, memory 406 included in the computing device 400 can store computer-readable and computer-executable instructions or software for implementing exemplary embodiments. Computing device 400 also includes processor 402 and associated core 404, and optionally, one or more additional processor(s) 402′ and associated core(s) 404′ (for example, in the case of computer systems having multiple processors/cores), for executing computer-readable and computer-executable instructions or software stored in the memory 406 and other programs for controlling system hardware. Processor 402 and processor(s) 402′ can each be a single core processor or multiple core (404 and 404′) processor.

Virtualization can be employed in computing device 400 so that infrastructure and resources in the computing device can be shared dynamically. A virtual machine 414 can be provided to handle a process running on multiple processors so that the process appears to be using only one computing resource rather than multiple computing resources. Multiple virtual machines can also be used with one processor.

Memory 406 can include a computer system memory or random-access memory, such as DRAM, SRAM, EDO RAM, and the like. Memory 406 can include other types of memory as well, or combinations thereof. An individual can interact with the computing device 400 through a visual display device/graphical user interface (GUI) 418, such as a touch screen display or computer monitor, which can display one or more user interfaces 422 for displaying data to the individual. The visual display device 418 can also display other aspects, elements and/or information or data associated with exemplary embodiments. The computing device 400 can include other input devices and I/O devices for receiving input from an individual, for example, a keyboard, a scanner, or another suitable multi-point touch interface 408, a pointing device 410 (e.g., a pen, stylus, mouse, or trackpad). The keyboard 408 and the pointing device 410 can be coupled to the visual display device 418. The computing device 400 can include other suitable conventional I/O peripherals.

The computing device 400 can also include one or more storage devices 424, such as a hard-drive, CD-ROM, or other computer readable media, for storing data and computer-readable instructions and/or software that implements exemplary embodiments of the system as described herein, or portions thereof. Exemplary storage device 424 can also store one or more databases for storing suitable information required to implement exemplary embodiments. The databases can be updated by an individual or automatically at a suitable time to add, delete or update data in the databases. Exemplary storage device 424 can store datasets 426, software 428, and other data/information used to implement exemplary embodiments of the systems and methods described herein.

The computing device 400 can include a network interface 412 configured to interface via one or more network devices 420 with one or more networks, for example, Local Area Network (LAN), Wide Area Network (WAN) or the Internet through a variety of connections including, but not limited to, standard telephone lines, LAN or WAN links (for example, 802.11, T1, T3, 56 kb, X.25), broadband connections (for example, ISDN, Frame Relay, ATM), wireless connections, processing device area network (CAN), or some combination of any or all of the above. The network interface 412 can include a built-in network adapter, network interface card, PCMCIA network card, card bus network adapter, wireless network adapter, USB network adapter, modem or another device suitable for interfacing the computing device 400 to a type of network capable of communication and performing the operations described herein. Moreover, the computing device 400 can be a computer system, such as a workstation, desktop computer, server, laptop, handheld computer, tablet computer (e.g., the iPad® tablet computer), mobile computing or communication device (e.g., the iPhone® communication device), or other form of computing or telecommunications device that is capable of communication and that has sufficient processor power and memory capacity to perform the operations described herein.

The computing device 400 can run an operating system 416, such as versions of the Microsoft® Windows® operating systems, the different releases of the Unix and Linux operating systems, a version of the MacOS® for Macintosh computers, an embedded operating system, a real-time operating system, an open source operating system, a proprietary operating system, an operating systems for mobile computing devices, or another operating system capable of running on the computing device and performing the operations described herein. In exemplary embodiments, the operating system 416 can be run in native mode or emulated mode. In an exemplary embodiment, the operating system 416 can be run on one or more cloud machine instances.

### INCORPORATION BY REFERENCE

The entire disclosure of each of the patent documents, including patent application documents, scientific articles, governmental reports, websites, and other references referred to herein is incorporated by reference herein in its entirety for all purposes. In case of a conflict in terminology, the present specification controls. All sequence listings, or Seq. ID. Numbers, disclosed herein are incorporated herein in their entirety.

The cited references, to the extent that they provide exemplary procedural or other details supplementary to those set forth herein, are specifically incorporated herein by reference.

Although illustrative embodiments of the present invention have been described herein, it should be understood that the invention is not limited to those described, and that various other changes or modifications may be made by one skilled in the art without departing from the scope or spirit of the invention.

1. A method for identifying a subject diagnosed with cancer or suspected of having cancer as falling within a treatable prognostic group, the method comprising:
a) providing a biological sample from the subject;
b) determining copy number of one or more target genes or fragments of the target genes compared to a reference sample by:
1) preparing a cell-free DNA (cfDNA) sample from the biological sample of step a);
2) preparing a sequencing library from the cfDNA from the biological sample, wherein preparing the library comprises consecutive steps of fragmenting the cfDNA, end-repairing, dA-tailing and adaptor ligating the cfDNA fragments;
3) sequencing the adaptor-ligated cfDNA fragments of the one or more target genes or fragments thereof from the biological sample;
4) determining copy number of the one or more target genes or fragments thereof from the sequences of the adaptor-ligated cfDNA fragments from the biological sample;
5) comparing the copy number of the one or more target genes or cfDNA fragments thereof for the biological sample with that of a reference sample, wherein the comparison can determine one or more chromosomal abnormalities in the biological sample;
c) identifying the prognostic group of the subject based on step b), wherein the presence of one or more chromosomal abnormalities in the one or more target genes or fragments thereof of the biological sample when compared to the reference sample indicates a subject having cancer with an adverse, intermediate, or favorable prognosis; and
d) qualifying the subject for chemotherapy or immunotherapy where the results in (c) indicate an adverse, intermediate or favorable prognosis, and
wherein the subject is human.

2. The method of claim 1, wherein the biological sample is
a) a tissue biopsy of the cancer or a liquid biopsy; or
b) blood, plasma, serum, urine, stool, saliva, tissue, or bodily fluid, or
c) plasma derived from peripheral blood that comprises a mixture of cfDNA derived from normal and cancerous cells.

3. (canceled)

4. (canceled)

5. The method of claim 1, wherein the one or more target genes or fragments thereof
a) are selected from Table 3; and/or
b) are protein-coding regions of a gene.

6. (canceled)

7. The method claim 1, wherein sequencing the adaptor-ligated cfDNA fragments of the one or more target genes or fragments thereof from the biological sample includes employing a next generation sequencing (NGS) method.

8. The method of claim 1,
a) wherein the cancer is selected from the group consisting of renal carcinoma, colorectal carcinoma, skin cancer, myelodysplastic syndrome (MDS), leukemia, lymphoma, myeloma, tumors of the central nervous system, breast cancer, prostate cancer, cervical cancer, uterine cancer, lung cancer, ovarian cancer, testicular cancer, thyroid cancer, astrocytoma, glioma, pancreatic cancer, mesotheliomas, gastric cancer, liver cancer, renal cancer including nephroblastoma, bladder cancer, oesophageal cancer, cancer of the larynx, cancer of the parotid, cancer of the biliary tract, endometrial cancer, adenocarcinomas, small cell carcinomas, neuroblastomas, adrenocortical carcinomas, epithelial carcinomas, desmoid tumors, desmoplastic small round cell tumors, endocrine tumors, Ewing sarcoma family tumors, germ cell tumors, hepatoblastomas, hepatocellular carcinomas, non-rhabdomyosarcome soft tissue sarcomas, osteosarcomas, peripheral primitive neuroectodermal tumors, retinoblastomas, and rhabdomyosarcomas, or
b) wherein the cancer is a myeloma or lymphoma.

9. (canceled)

10. The method of claim 1, wherein the chromosomal abnormalities in the one or more target genes or fragments thereof is a deletion, a duplication, or a combination thereof.

11. (canceled)

12. The method of claim 1, wherein step b-4) comprises determining sequence read depth of one or more target segments and one or more non-target segments.

13. The method claim 1, wherein the reference sample is a sample from one or more subjects with a cancer or a sample from one or more subjects not with a cancer.

14. The method of claim 1, wherein qualifying the subject for chemotherapy or immunotherapy comprise identifying the subject as a candidate for chemotherapy or immunotherapy based on the adverse, intermediate or favorable prognosis.

15. The method of claim 1, wherein qualifying the subject for chemotherapy or immunotherapy comprises one or more of:
displaying on a graphical user interface an identification of the subject as a candidate for chemotherapy or immunotherapy;
storing data identifying the subject as a candidate for chemotherapy or immunotherapy;
sending an electronic communication including an identification of the subject as a candidate for chemotherapy or immunotherapy;
displaying on a graphical user interface a recommendation of chemotherapy or immunotherapy for the subject;
storing data including a recommendation of immunotherapy or chemotherapy for the subject; and
sending an electronic communication including a recommendation of immunotherapy or chemotherapy for the subject.

16. The method of claim 1,
a) wherein identifying the prognostic group of the subject as adverse includes the biological sample comprising one or more chromosomal abnormalities selected from the group consisting of t(6;9)(p23;q34); DEK-NUP214; t(v;11q23); KMT2A rearranged; t(9;22)(q34;q11); BCR-ABL1; inv(3)(q21q26) or t(3;3)(q21;q26); GATA2,MECOM(EVI1); del(5q); abn(17p); complex karyotype; and monosomal karyotype;
b) wherein identifying the prognostic group of the subject as favorable includes the biological sample comprising one or more chromosomal abnormalities selected from the group consisting of t(8;21)(q22;q22); RUNX1-RUNX1T1; inv(16)(p13q22) or t(16;16)(p13;q22); CBFB-MYH11; and 415;17) PML-RARA; and does not include one or more chromosomal abnormalities selected from the group consisting of t(9;11)(p21;q23); MLLT3-KMT2A; t(6;9)(p23;q34); DEK-NUP214; t(v;11q23); KMT2A rearranged; t(9;22)(q34;q11); BCR-ABL1; inv(3)(q21q26) or t(3;3)(q21;q26); GATA2,MECOM(EVI1); del(5q); abn(17p); Complex karyotype; and monosomal karyotype; and
c) wherein identifying the prognostic group of the subject as intermediate includes the biological sample comprising one or more chromosomal abnormalities selected from the group consisting of t(9;11)(p21;q23); MLLT3-KMT2A; and does not include t(8;21)(q22;q22); RUNX1-RUNX1T1; inv(16)(p13q22) or t(16;16)(p13;q22); CBFB-MYH11; t(15;17) PML-RARA; t(6;9)(p23;q34); DEK-NUP214; t(v;11q23); KMT2A rearranged; t(9;22)(q34;q11); BCR-ABL1; inv(3)(q21q26) or t(3;3)(q21;q26); GATA2,MECOM(EVI1); del(5q); abn(17p); Complex karyotype; and monosomal karyotype.

17. (canceled)

18. (canceled)

19. A method for treating a subject with a cancer, the method comprising:
a) providing a biological sample from the subject;
b) determining copy number of one or more target genes or fragments of the target genes compared to a reference sample by:
1) preparing a cell-free DNA (cfDNA) sample from the biological sample of step a);
2) preparing a sequencing library from the cfDNA from the biological sample, wherein preparing the library comprises consecutive steps of fragmenting the cfDNA, end-repairing, dA-tailing and adaptor ligating the cfDNA fragments;
3) sequencing the adaptor-ligated cfDNA fragments of the one or more target genes or fragments thereof from the sequences of the adaptor-ligated cfDNA fragments for the biological sample and determining copy number of the one or more target genes or fragments thereof;
4) comparing, by a computing system, the copy number of the one or more target genes or cfDNA fragments thereof for the biological sample with that of a reference sample, wherein the comparison can determine one or more chromosomal abnormalities in the biological sample;
c) identifying the prognostic group of the subject based on step b), wherein the presence of one or more chromosomal abnormalities in the one or more target genes or fragments thereof for the biological sample when compared to the reference sample indicates a subject has a cancer with an adverse, intermediate, or favorable prognosis;
d) qualifying the subject for adjunct therapy where the results in (c) indicate an adverse, intermediate or favorable prognosis, and
e) administering a therapeutic amount of a chemotherapeutic or immunotherapeutic agent to the subject qualified for adjunct therapy in the adverse, intermediate or favorable prognosis group, and
wherein the subject is human.

20. The method of claim 19, wherein the biological sample is
a) a tissue biopsy of the cancer or a liquid biopsy; or
b) blood, plasma, serum, urine, stool, saliva, tissue, or bodily fluid, or
c) plasma derived from peripheral blood that comprises a mixture of cfDNA derived from normal and cancerous cells.

21. (canceled)

22. (canceled)

23. The method of claim 19, wherein the one or more target genes or fragments thereof
a) are selected from Table 3; and/or
b) is a protein-coding region of a gene.

24. (canceled)

25. The method of claim 19, wherein sequencing the adaptor-ligated cfDNA fragments of the one or more target genes or fragments thereof from the biological sample includes employing a next generation sequencing (NGS) method.

26. The method of claim 19,
a) wherein the cancer is selected from the group consisting of renal carcinoma, colorectal carcinoma, skin cancer, myelodysplastic syndrome (MDS), leukemia, lymphoma, myeloma, tumors of the central nervous system, breast cancer, prostate cancer, cervical cancer, uterine cancer, lung cancer, ovarian cancer, testicular cancer, thyroid cancer, astrocytoma, glioma, pancreatic cancer, mesotheliomas, gastric cancer, liver cancer, renal cancer including nephroblastoma, bladder cancer, oesophageal cancer, cancer of the larynx, cancer of the parotid, cancer of the biliary tract, endometrial cancer, adenocarcinomas, small cell carcinomas, neuroblastomas, adrenocortical carcinomas, epithelial carcinomas, desmoid tumors, desmoplastic small round cell tumors, endocrine tumors, Ewing sarcoma family tumors, germ cell tumors, hepatoblastomas, hepatocellular carcinomas, non-rhabdomyosarcome soft tissue sarcomas, osteosarcomas, peripheral primitive neuroectodermal tumors, retinoblastomas, and rhabdomyosarcomas, or
b) wherein the cancer is a myeloma or lymphoma.

27. (canceled)

28. The method of claim 19, wherein the chromosomal abnormality is a deletion, a duplication, or a combination thereof.

29. (canceled)

30. The method of claim 19, wherein step b-3) comprises determining sequence read depth of one or more target segments and one or more non-target segments.

31. The method of claim 19, wherein the reference sample is a biological sample from one or more subjects with cancer or a biological sample from one or more subjects not with cancer.

32. The method of claim 19,
a) wherein identifying the prognostic group of the subject as adverse includes the biological sample comprising one or more chromosomal abnormalities selected from the group consisting of t(6;9)(p23;q34); DEK-NUP214; t(v;11q23); KMT2A rearranged; t(9;22)(q34;q11); BCR-ABL1; inv(3)(q21q26) or t(3;3)(q21;q26); GATA2,MECOM(EVI1); del(5q); abn(17p); Complex karyotype; and monosomal karyotype,
b) wherein identifying the prognostic group of the subject as favorable includes the biological sample comprising one or more chromosomal abnormalities selected from the group consisting of t(8;21)(q22;q22); RUNX1-RUNX1T1; inv(16)(p13q22) or t(16;16)(p13;q22); CBFB-MYH11; and 415;17) PML-RARA; and does not include one or more of chromosomal abnormalities selected from the group consisting of t(9;11)(p21;q23); MLLT3-KMT2A; t(6;9)(p23;q34); DEK-NUP214; t(v;11q23); KMT2A rearranged; t(9;22)(q34;q11); BCR-ABL1; inv(3)(q21q26) or t(3;3)(q21;q26); GATA2,MECOM(EVI1); del(5q); abn(17p); Complex karyotype; and monosomal karyotype; and
c) wherein identifying the prognostic group of the subject as intermediate includes the biological sample comprising one or more chromosomal abnormalities selected from the group consisting of t(9;11)(p21;q23); MLLT3-KMT2A; and does not include t(8;21)(q22;q22); RUNX1-RUNX1T1; inv(16)(p13q22) or t(16;16)(p13;q22); CBFB-MYH11; t(15;17) PML-RARA; t(6;9)(p23;q34); DEK-NUP214; t(v;11q23); KMT2A rearranged; t(9;22)(q34;q11); BCR-ABL1; inv(3)(q21q26) or t(3;3)(q21;q26); GATA2,MECOM(EVI1); del(5q); abn(17p); Complex karyotype; and monosomal karyotype.

33. (canceled)

34. (canceled)
